(19) United States
(12) Patent Application Publication
(10) Pub. No.: US 2026/0364052 A1
(43) Pub. Date: Nov. 19, 2026

(54) Stabilized N-substituted Indole Carbinols and Their Use as Anti-Tumor Prodrugs

(71) Applicant: Fannin Partners, LLC, HOUSTON, TX (US)

(72) Inventors: Dev Kumar Chatterjee, Nashville, TN (US); Atul Varadhachary, Bellaire, TX (US); Uksha Saini, Pearland, TX (US); Leniher Castan Chibas, Richmond, TX (US); Ross Hamilton, Houston, TX (US); Zebib Sielu Abraha, Richmond, TX (US); Stephanie Vega, Houston, TX (US)

(21) Appl. No.: 19/575,660

(22) Filed: Mar. 23, 2026

Related U.S. Application Data
(60) Provisional application No. 63/781,137, filed on Mar. 31, 2025.
(60) Provisional application No. 63/809,001, filed on May 20, 2025.

(51) Int. Cl. A61K 31/4045; A61K 31/404; A61K 47/14; A61K 47/16; A61K 47/40; A61P 35/00

(52) U.S. Cl. CPC A61K 31/4045; A61K 31/404; A61K 47/14; A61K 47/16; A61K 47/40; A61P 35/00

(57) ABSTRACT

Disclosed are stable N-substituted indole carbinol compounds, compositions including them, including pharmaceutical formulations thereof; and the use of such compositions and formulations in mammalian therapy, including in treatment of human tumors and cancers. In particular, three such N-substituted indole carbinol compounds were found efficacious against tumors in in vitro and in vivo tests.

### FIELD

The invention relates to stabilized heterocyclyl compounds, including indolyl compounds (e.g., N-substituted indole carbinols), and their use in therapies for cancers.

### BACKGROUND

The sulfotransferase 1 (SULT1) family belongs to the superfamily sulfotransferase enzymes that play an important role in the metabolism of numerous compounds, including xenobiotics, drugs and certain endogenous compounds. They are normally found in some organs and cells of the body and act as a major metabolic/detoxification system. They are highly expressed in tissues that are exposed to foreign compounds: including the lungs and respiratory tract (including the nasal cavity), the gastrointestinal tract, and the liver.

A major role of sulfonation in the body is the metabolism and detoxification of various xenobiotic compounds via conjugation with a sulfonate moiety, which makes the compounds more soluble at physiological pH and easier to excrete. A variety of xenobiotics are substrates for different SULT1 enzymes in the intestines and liver, including (−)-salbutamol, 7-OH-flavone, paracetamol, and (−)-apomorphine.

Sulfonation also plays a strong regulatory role for several classes of endogenous substrates, including estrone, cholesterol, dopamine, bile acid salts, testosterone, and neuroendocrine peptide cholecystokinin (CCK). For example, in normal human plasma, some estimates are that 99% of total dopamine, 78% of total noradrenaline, and 67% of total adrenaline are present in inactive sulfonated form. Eisenhofer et al., Dopamine sulphate: an enigma resolved. Clin Exp Pharmacol Physiol Suppl. 1999 April; 26:S41-53. PMID: 10386253. Similarly, the level of the inactive sulfonated form of the hydroxysteroid hormone dehydroepiandrosterone (DHEA) in plasma has been shown to be about 100-fold higher than the level of unsulfonated DHEA. Falany J L, Falany C N, Regulation of estrogen activity by sulfation in human MCF-7 breast cancer cells. Oncol Res. 1997; 9(11-12):589-96. PMID: 9563006. Sulfonation can also increase activity of endogenous molecules, as in the case of the neuroendocrine peptide cholecystokinin (CCK), which exhibits biological activity when in sulfated form. Vargas et al., 3′-Phosphoadenosine 5′-phosphosulfate biosynthesis and the sulfation of cholecystokinin by the tyrosylprotein-sulfotransferase in rat brain tissue. Chem Biol Interact. 1994 June; 92(1-3):281-91. doi: 10.1016/0009-2797(94)90070-1. PMID: 8033261.

All members of SULT1 family can catalyze some of the same substrates, including phenolic compounds like p-nitrophenol. Usually, addition of a sulfonate moiety to a compound increases its solubility because sulfates are fully ionized at physiological pH; but such addition usually also decreases biological activity. These enzymes are also capable of activating procarcinogens to reactive electrophiles. The universal sulfonate donor for the sulfonations catalyzed by sulfotransferases is 3′-phospho-adenosyl-5′-phosphosulfate (PAPS). In humans, SULT1 participates in the phase II metabolism of drugs (i.e., conversion to polar inactive metabolites).

A variety of drugs and natural products are also inhibitors of these compounds. For example, several natural compounds that exhibit antioxidant and anticancer activity, such as curcumin from curry powder and epigallocatechins from green tea, are potent inhibitors of phenolic sulfotransferases. The protective role of these sulfotransferase inhibitors is likely due to the involvement of sulfotransferases in the activation of several pro-carcinogenic compounds to fully activated carcinogens. This happens when hydroxyl groups in allylic or benzylic alcohols, or in hydroxylamines formed from aromatic amines, get sulfonated. The resulting sulfates are unstable and readily decompose to inorganic sulfate and highly reactive electrophilic carbonium or nitrenium cations. These species can then attack highly nucleophilic DNA and covalently bind to it, which may lead to mutagenesis and carcinogenesis. Similarly, several drugs or their metabolites having phenolic hydroxyl groups, including the cancer drugs raloxifene and tamoxifen, are also metabolized predominantly through sulfonation.

Sulfotransferases can be divided into two large groups: the cytosolic SULTs and the membrane-associated SULTs, which are localized in the Golgi apparatus. Cytosolic SULTs catalyze the sulfonation of xenobiotics, hormones, monoamine neurotransmitters, and drugs. Orally administered xenobiotics and drugs are to a large extent sulfonated in the intestines and in the liver, and excreted either via urine or bile. Membrane-associated SULTs catalyze the sulfonation of proteins and carbohydrates such as glycosaminoglycans, glycoproteins, for processes such as cellular signaling and modulation of receptor binding. Recent studies have implicated the SULTs in a number of disease states, including entry of herpes virus (Xu ea al., Characterization of heparan sulphate 3-O-sulphotransferase isoform 6 and its role in assisting the entry of herpes simplex virus type 1. Biochemical Journal 385.2 (2005): 451-459) entry of HIV (Seibert et al., Tyrosine sulfation of CCR5 N-terminal peptide by tyrosylprotein sulfotransferases 1 and 2 follows a discrete pattern and temporal sequence. Proc Natl Acad Sci USA. 2002 Aug. 20; 99(17):11031-6. doi: 10.1073/pnas.172380899. Epub 2002 Aug. 8. PMID: 12169668; PMCID: PMC123205), chronic inflammation (A. van Zante, S. D. Rosen; Sulphated endothelial ligands for L-selectin in lymphocyte homing and inflammation. Biochem Soc Trans 1 Apr. 2003; 31 (2): 313-317. doi: https://doi.org/10.1042/bst0310313), and cancer (Suzuki et al., Genetic polymorphisms of estrogen receptor alpha, CYP19, catechol-O-methyltransferase are associated with familial prostate carcinoma risk in a Japanese population. Cancer, 98: 1411-1416. https://doi.org/10.1002/cncr.11639).

The SULT1 family includes 3 subfamilies: SULT1A, SULT1B and SULT1C, each of which consists of different isoforms. SULT1A is the largest subfamily with 4 isoforms (SULT1A1, A2, A3 and A4) that are >90% identical in their genetic coding sequences. SULT1A sulfotransferases are the most abundant and widespread. These differ based on their metabolic preference for different substrates: phenol sulfotransferase SULT1A1, and monoamine sulfotransferase SULT1A3.

The interaction between sulfotransferases and carcinogens, i.e. activation and inactivation of carcinogens by sulfotransferases, has been extensively studied. Glatt, Hansruedi. “13 Activation and Inactivation of Carcinogens and Mutagens by Human Sulfotransferases.” Human Cytosolic Sulfotransferases (2005): 279. A majority of the pro-carcinogens are aromatic compounds, which can be easily functionalized in such manner that they contain either a benzylic or allylic alcohol or an aromatic hydroxylamine structural unit. The hydroxy group is readily sulfonated by the sulfotransferases, yielding activated sulfuric acid esters of benzylic and allylic alcohols, and aromatic hydroxylamines, which readily lose the sulfate moiety to form resonance-stabilized carbonium or nitrenium ions. These reactive electrophilic species then react with nucleophilic sites on DNA, leading to mutagenicity and carcinogenicity.

Further, the addition of a sulfonate moiety mediated by SULT1 family can also turn an inactive compound into a reactive electrophile. It is reported that the SULT1A1 isoform activates the N-Benzyl-Indole-Carbinol (N-BIC) class of compounds, converting them from an inactive prodrug form to a highly reactive electrophilic form that covalently binds to proteins and/or DNA, leading eventually to cell death. This can be useful to create anticancer drugs that are inactive until activated by the SULT1A1 enzyme. These drugs can be used wherever there is excessive expression of the enzyme. SULT1A1 is over-expressed in up to 15% of major cancer types, including but not limited to breast, lung, prostate, kidney, pancreatic, liver and brain cancer. Such high expression of SULT1A1, identified at mRNA and/or protein level, can be exploited for tumor killing by N-BIC.

N-Benzyl Indole Carbinols (N-BICs) are synthetic derivatives of a natural product, indole-3-carbinol (I3C), commonly found in cruciferous vegetables. I3C, an acid-sensitive compound, was reported to have anticancer activity in different types of cancer. N-BICs differ from I3C in having a benzyl group off the indole nitrogen, which gives them additional chemical stability over I3C under acidic conditions. N-BICs have demonstrated potent anti-tumor effects in a number of solid- and liquid-tumor types where there is also significant expression of SULT1A1. Independent studies have shown that SULT1A1 expression strongly correlates with sensitivity to N-BICs in different cancer types.

Despite the potential antitumor efficacy in different types of cancer, development of N-BIC analogs has lagged due to solubility and stability issues, preventing their advancement to the clinic. Thus, what is needed for their use in therapy, is a stable and soluble formulation of N-BICs and/or related compounds with the same activity.

### SUMMARY

The present invention includes stable N-substituted indole carbinol compounds, compositions including them, including pharmaceutical formulations thereof; and further includes the use of such compositions and formulations in mammalian therapy, including in treatment of human tumors and cancers. A generic formula for such compounds and formulations is:

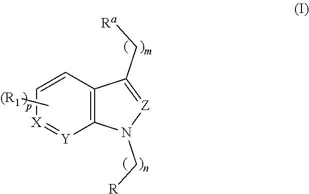

where X, Y, and Z are independently either N or CH; R optionally substituted alkyl, optionally substituted cycloalkyl, optionally substituted heteroalkyl, optionally substituted heterocyclyl, optionally substituted aryl, optionally substituted heteroaryl, optionally substituted carbamido, optionally substituted alkamino, optionally substituted sulfonyl, and optionally substituted N-sulfonamido; n is 0, 1, 2, or 3; R1 is selected from a group consisting of optionally substituted alkyl, halogen, and a hydrogen isotope; p is 0, 1, 2, 3, or 4; Ra is selected from a group consisting of hydroxyl, optionally substituted alkyl, optionally substituted cycloalkyl, optionally substituted aryl, optionally substituted heteroaryl, and optionally substituted heteroalkyl; and m is 0, 1, 2, or 3.

Several embodiments pertain to compositions comprising compounds of the generic formula above administered with a cyclodextrin. In several embodiments, the cyclodextrin is a β-cyclodextrin or a γ-cyclodextrin. In several embodiments, the β-cyclodextrin is methyl-β-cyclodextrin. In several embodiments, the β-cyclodextrin is Captisol® (is sulfobutylether-β-cyclodextrin). In several embodiments, the composition comprises a carrier.

Several embodiments pertain to a method of treating a disease in a subject. In several embodiments, the method comprising administering to said subject a composition or compound as disclosed herein. In several embodiments, the disease is a cancer. In several embodiments, the cancer is selected from a group consisting of breast cancer, lung cancer, prostate cancer, kidney cancer, pancreatic cancer, liver cancer, brain cancer, ovarian cancer, colon cancer, leukemia, lymphoma, multiple myeloma, thyroid cancer, bone cancer, and esophageal cancer. In several embodiments, the compound is administered parenterally, orally, subcutaneously, topically, rectally, intrathecally, intracerebrally, intramuscularly, epidurally, intravenously, intraperitoneally, vaginally, or pulmonarily. Several embodiments herein disclose stable N-substituted indole carbinol compounds, compositions comprising the same, including pharmaceutical formulations thereof, and the use of such compositions and formulations in mammalian therapy, including in treatment of human tumors and cancers. Also described herein are companion diagnostics to identify patients who will benefit most from such therapy, such as, for example, patients expressing one or more than one member of the SULT1 family at levels exceeding a threshold.

A preferred composition can include the two compounds shown below, respectively designated (left-side compound) as BST-10, the IUPAC name being: 3-(Hydroxymethyl)-1-[2-(1H-pyrrol-1-yl)ethyl]-1H-indole, and BST-12 (right-side compound), the IUPAC name being: 3-(Hydroxymethyl)-5-methoxy-1-(4-methoxybenzyl)-1H-indole.

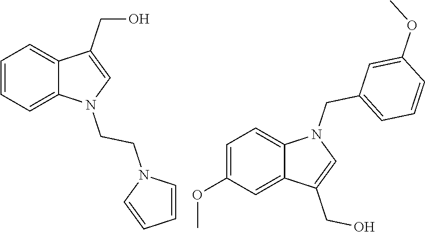

BST-10 and BST-12 may be administered with inclusion of β-cyclodextrin or γ-cyclodextrin, or another cyclodextrin. Another preferred composition includes the compound below, having the IUPAC name 3-(Hydroxymethyl)-1-(4-methoxybenzyl)-1H-indole, and designated Compound 103:

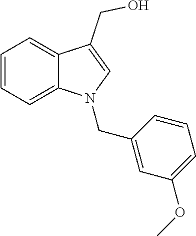 [SMILES: COc1cccc(Cn2cc(CO)c3ccccc32)c1]

Compositions of the invention for therapeutic use with BST-10, BST-12 and Compound 103 may, in addition to cyclodextrin, also include polyethylene glycol (15)-hydroxystearate (also called Solutol HS15) which is an amphiphilic nonionic surfactant, as well as pharmaceutically acceptable excipients, including hydrophilic excipients.

Also described are companion diagnostics to identify patients who will benefit most from such therapy; for example, patients expressing one or more than one members of the SULT1 family at levels exceeding a threshold, as determined from protein and/or mRNA levels in patient samples.

The above-mentioned N-substituted Indole Carbinol compositions can be used for therapeutic treatment for diseases including cancers, e.g., breast cancer, lung cancer, prostate cancer, kidney cancer, pancreatic cancer, liver cancer, brain cancer, ovarian cancer, colon cancer, leukemia, lymphoma, multiple myeloma, thyroid cancer, bone cancer, and esophageal cancer.

The compositions of the invention can be administered parenterally, orally, subcutaneously, topically, rectally, intrathecally, intracerebrally, intramuscularly, epidurally, intravenously, intraperitoneally, vaginally, or pulmonarily. The compositions can be administered all at once, multiple times, or delivered substantially uniformly over a period of time. It is also noted that the dosage can be varied over time.

For human or large mammal therapy, the dosage can be found by extrapolation from animal experiments, by multiplying the active ingredient dosage found effective in the animal by the weight proportion of the human or mammal subject to whom the composition is to be administered and the animal.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts evaluation of Compound 103 antitumor activity in a relative cell viability assay (Panel B) with 4 RCC cell lines whose SULT1A1 expression, as shown in panel A where the upper panel is a Western blot with B-Actin as a control, and the lower panel shows SULT1A1 mRNA expression, and the results from panel A show that A498 and Caki display high SULT1A1 expression, and 786-O and ACHN display low SULT1A1 expression. FIG. 1B depicts a tumor regression test using a A498 subcutaneous xenograft mouse model injected IP daily for 14 days with a preferred Compound 103 formulation, either 25 mg/kg or 50 mg/kg for two days, followed by three days with no drug, then 10 mg/kg thereafter. The Y axis represents the average relative weight of the mice in each treatment (or non-treatment) group.

FIG. 2 shows two representative structures of SULT1A1—as shown in FIGS. 3A and 3B, aligned and superimposed, where the simulation with 1IS6 (see below) is in green and with 2D06, is in grey.

FIG. 3A shows the cluster representative structure of Compound 103 during 100 ns Molecular Dynamics (MD) simulation with 2D06 (where 2D06 represents the crystal structure of the Human Estrogen Receptor Alpha (ERα) ligand-binding domain in complex with Compound 103); and FIG. 3B shows the structure during 100 ns MD simulation with 1LS6 (Human Serum Albumin complexed with myristic acid).

FIG. 4 shows the starting Compound 103 backbone and R1 attachment point for generating additional analogue and homologue compounds.

FIG. 5 shows structures of 50 novel compounds which are Compound 103 analogs identified by a computational chemistry approach.

FIG. 6 shows the structures of five N-substituted Indole Carbinols designed based on a medicinal chemistry approach.

FIG. 7 shows synthesis routes for seven of the 58 analogs of primary interest.

FIG. 8 shows the initial stability analysis of Compound 103 after 24 hours at either 2-8 °C or room temperature. Water and PBS systems contained 30% acetonitrile with 1.9 mM Compound 103. CD solutions contained 12.9 mM Compound 103 and 310 mg/m of cyclodextrin (CD), which were either 2-hydroxypropyl-beta-cyclodextrin (HPPCD), hydroxypropyl-x-cyclodextrin (HPGCD), methyl-β-cyclodextrin (MBCD) or sulfobutylether-β-cyclodextrin (SBEBCD).

FIG. 9 shows stability of 12.9 mM Compound 103 in 310 mg/ml CD solutions after 24 hours at different temperatures and at different temperatures in PBS (either 2-8 °C or room temperature), where the Y-axis shows the percentage of Compound 103 recovered.

FIG. 10 shows stability of two different concentrations of Compound 103 in two different concentrations of MβCD over 50 days at either 2-8 °C or room temperature.

FIG. 11 shows the stability of formulations of different Compound 103 concentrations in different HPPCD over 35 days at either 2-8 °C or room temperature.

FIG. 12A shows tumor growth in a mouse model control group of five mice over 20 days. 12B shows tumor growth in a mouse model group of five mice over 41 days, where mice were administered 25 mg/kg of a composition of Compound 103.

FIG. 12C shows tumor growth in a mouse model group of five mice over 41 days, where mice were initially administered 50 mg/kg of a composition of Compound 103 for two days, administration was stopped for three days, and then administration was continued at 10 mg/kg for the remainder of the study. FIG. 12C shows that all mice administered Compound 103 in the mouse kidney tumor model had non-detectable after 14 days that remained absent through day 41.

FIG. 13A shows using NCI-60 screening to identify cell lines susceptible to BST10 cytotoxicity.

FIG. 13B shows using NCI-60 screening to identify cell lines susceptible to BST12 cytotoxicity.

FIG. 14A shows that BST12 selectively inhibits growth of SULT1A1-positive NCI-60 cell lines.

FIG. 14B, shows that BST10 selectively inhibits growth of SULT1A1-Positive NCI-60 cell lines.

FIG. 15A shows that BST12 selectively kills SULT1A1-positive cancer cells.

FIG. 15B, shows that BST10 selectively kills SULT1A1-positive cancer cells.

FIG. 15C shows a Western blot summary table (DepMap and western blot), where FIG. 15D was the Western blot source.

FIG. 15D shows a Western blot verifying SULT1A1 expression in the tested cell lines.

FIGS. 16A and 16B show that BST12 demonstrates minimal in vivo toxicity in Sprague Dawley rats.

FIG. 17 (first panel) shows a sulfonation assay using recombinant SULT1A1 (R&D Systems, Minneapolis, MN), wherein Compound 103 (0.5 mM) is sulfonylated by SULT1A1 in the presence of p-nitrophenyl sulfate (sulfonate donor) and 5′-phosphoadenosine-3′-phosphosulfate (PAPS), the cofactor used by sulfotransferases such as SULT1A1 to transfer a sulfate group to the substrate. In the second panel in FIG. 17, 2-Naphthol (0.1 mM) a known substrate of SULT1A1, serves as a positive control. The test was run under similar conditions using the recombinant SULT1A1.

FIG. 18 shows that Compound 103 demonstrates potent antitumor activity in a SULT1A1-positive CDx mouse model.

### DETAILED DESCRIPTION

Several embodiments disclosed herein pertain to substituted indolyl compounds or a N-substituted indole comprising an alkanol (e.g., carbinol), methods of using these compounds in therapy, and compositions including these compounds. The compounds are preferably suitable for use in targeting sulfotransferase enzyme related diseases or disorders, and are believed to act as sulfotransferase enzyme antagonists. In several embodiments, the indolyl compound comprises a cyclic substituent (e.g., an optionally substituted aryl, heteroaryl, etc.) bonded to the N of the indole ring (e.g., at the 1-position). In several embodiments, the indolyl compound is optionally substituted at the 3-position. In several embodiments, the indolyl compound is optionally substituted on any one or more of the 4, 5, 6, or 7 positions. In several embodiments, the indolyl compound lacks a substituent at the 2-position.

The following description and the figures referenced provides context and examples but should not be interpreted to limit the scope of the inventions. No single component or collection of components is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

### DEFINITIONS

The section headings used herein are for organizational purposes only and are not to be construed as limiting the subject matter described. Unless defined otherwise, all technical and scientific terms used herein have the same meaning as is commonly understood by one of ordinary skill in the art. All patents, applications, published applications, and other publications are incorporated by reference in their entirety. In the event that there is a plurality of definitions for a term herein, those in this section prevail unless stated otherwise.

A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound which is administered as an ester (the “prodrug”) to facilitate transmittal across a cell membrane where water solubility is detrimental to mobility, but which is then metabolically hydrolyzed to the carboxylic acid (the active entity) once inside the cell where water solubility is beneficial. A further example of a prodrug might be a short peptide (polyaminoacid) bonded to an acid group where the peptide is metabolized to reveal the active moiety. Conventional procedures for the selection and preparation of suitable prodrug derivatives are described, for example, in Design of Prodrugs, (Bundgaard, Hans. Design of Prodrugs. Amsterdam; New York: Elsevier; New York, NY, USA: Elsevier Science Pub. Co., 1985. Print.), which is hereby incorporated herein by reference in its entirety.

The term “pro-drug ester” refers to derivatives of the compounds disclosed herein formed by the addition of any of several ester-forming groups that are hydrolyzed under physiological conditions. Examples of pro-drug ester groups include pivoyloxymethyl, acetoxymethyl, phthalidyl, indanyl and methoxymethyl, as well as other such groups known in the art, including a (5-R-2-oxo-1,3-dioxolen-4-yl) methyl group. Other examples of pro-drug ester groups can be found, for example, in T. Higuchi and V. Stella, in “Pro-drugs as Novel Delivery Systems,” Vol. 14, A.C.S. Symposium Series, American Chemical Society (1975) and “Bioreversible Carriers in Drug Design: Theory and Application,” edited by E. B. Roche, Pergamon Press: New York, 14-21 (1987) (providing examples of esters useful as prodrugs for compounds containing carboxyl groups). Each of the above-mentioned references is herein incorporated by reference in their entirety.

The term “pharmaceutically acceptable carrier” or “pharmaceutically acceptable excipient” includes any and all solvents (including aqueous solutions), dispersion media, coatings, antibacterial and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990), Goodman and Gilman's: The Pharmacological Basis of Therapeutics, 8th Ed., Pergamon Press, which is incorporated herein by reference in its entirety.

“Metabolites” of the compounds disclosed herein include active species that are produced upon introduction of the compounds into the biological milieu.

“Solvate” refers to the compound formed by the interaction of a solvent and a compound described herein, a metabolite, or a salt thereof. Suitable solvates are pharmaceutically acceptable solvates including hydrates.

The term “pharmaceutically acceptable salt” refers to salts that retain the biological effectiveness and properties of a compound, and which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium, and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

When referring to numerical values, the term “or ranges including and/or spanning the aforementioned values” (and variations thereof) is meant to include any range that includes or spans the aforementioned values. For example, when the temperature of a reaction is expressed as “20 °C, 30 °C, 40 °C, 50 °C, or ranges including and/or spanning the aforementioned values,” this includes the particular temperature provided or temperature ranges spanning from 20 °C to 50 °C, 20 °C to 40 °C, 20 °C to 30 °C, 30 °C to 50 °C, 30 °C to 40 °C, or 40 °C or 50 °C.

As used herein, ““Ca to Cb” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, or heteroaryl group. That is, the alkyl, alkenyl, alkynyl, ring of the cycloalkyl, ring of the cycloalkenyl, ring of the cycloalkynyl, ring of the aryl, or the ring of the heteroaryl can contain from “a” to “b”, inclusive, carbon atoms. Thus, for example, a “C₁ to C₄ alkyl” group refers to all alkyl groups having from 1 to 4 carbons (e.g., 1, 2, 3, or 4), that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. A “C₁ to C₆ alkyl” group If no “a” and “b” are designated with regard to an alkyl, alkenyl, alkynyl, cycloalkyl cycloalkenyl, cycloalkynyl, aryl, or heteroaryl group, the ranges described in these definitions are included (including the broadest ranges).

As used herein, “alkyl” refers to a straight or branched hydrocarbon chain that is fully saturated (i.e., contains no double or triple bonds). The alkyl moiety may be branched or straight chain. Examples of branched alkyl groups include, but are not limited to, iso-propyl, sec-butyl, t-butyl, and the like. Examples of straight chain alkyl groups include, but are not limited to, methyl, ethyl, n-propyl, n-butyl, n-pentyl, n-hexyl, n-heptyl, and the like. The alkyl group may have 1 to 20 carbon atoms (as disclosed elsewhere herein, whenever it appears herein, a numerical range such as “1 to 20” refers to each integer in the given range; for example, “1 to 20 carbon atoms” means that the alkyl group may consist of 1, 2, 3, 4, 5, 6, 7, 8, 9, 10, 11, 12, 13, 14, 15, 16, 17, 18, 19, or 20 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. The alkyl group of the compounds may be designated as “C₁₋₆ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl (“Me” or —CH₃), ethyl, propyl, isopropyl, butyl, isobutyl, tertiary butyl, pentyl, hexyl, and the like. An alkyl group may be unsubstituted or substituted.

As used herein, “alkenyl” refers to an alkyl group that contains in the straight or branched hydrocarbon chain one or more double bonds. As noted in the definition of “alkyl,” an alkenyl group may be unsubstituted or substituted.

As used herein, “alkynyl” refers to an alkyl group that contains in the straight or branched hydrocarbon chain one or more triple bonds. As noted in the definition of “alkyl”, an alkynyl group may be unsubstituted or substituted.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene, and octylene. An alkylene group may be represented by, followed by the number of carbon atoms, followed by a “*”. For example, to represent ethylene. The alkylene group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. For example, a lower alkylene group can be substituted by replacing one or more hydrogen of the lower alkylene group and/or by substituting both hydrogens on the same carbon with a C₃-6 monocyclic cycloalkyl group, e.g.:

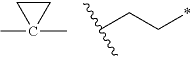

It is also to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.” An alkylene group may be substituted or unsubstituted.

As used herein, “alkanol” refers to a straight- or branched-chain alkyl group, substituting one or more hydroxyl groups. The alkanol may comprise a medium sized or lower alkyl. An alkanol group may be substituted or unsubstituted.

The term “halogen” or “halo,” as used herein, means any one of the radio-stable atoms of column 7 of the Periodic Table of the Elements, e.g., fluorine (—F), chlorine (—Cl), bromine (—Br), or iodine (—I).

As used herein, “haloalkyl” refers to a straight- or branched-chain alkyl group, substituting one or more hydrogens with halogens. Examples of haloalkyl groups include, but are not limited to, —CF₃, —CHF₂, —CH₂F, —CH₂CF₃, —CH₂CHF₂,—CH₂CH₂F, —CH₂CH₂C₁, —CH₂CF₂CF₃, and other groups that in light of the ordinary skill in the art and the teachings provided herein would be considered equivalent to any one of the foregoing examples. The haloalkyl may be a medium sized or lower haloalkyl. An haloalkyl group may be substituted or unsubstituted.

As used herein, “alkoxy” refers to the formula —OR wherein R is an alkyl as is defined above, such as “C1-9 alkoxy”, including but not limited to methoxy, ethoxy, n-propoxy, 1-methylethoxy (isopropoxy), n-butoxy, iso-butoxy, sec-butoxy, and tert-butoxy, and the like. An alkoxy group may be substituted or unsubstituted.

As used herein, “polyethylene glycol” refers to the formula

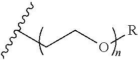

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain (e.g., alkyl) containing one or more heteroatoms. A heteroatom is given its plain and ordinary meaning in organic chemistry, which includes an element other than carbon, including but not limited to, nitrogen (e.g., amino, etc.), oxygen (e.g., alkoxy, ether, hydroxyl, etc.), sulfur, and halogens. The heteroalkyl group may have 1 to 20 carbon atoms although the present definition also covers the occurrence of the term “heteroalkyl” where no numerical range is designated. The heteroalkyl group may also be a medium size heteroalkyl having 1 to 12 carbon atoms. The heteroalkyl group could also be a lower heteroalkyl having 1 to 6 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C1-4 heteroalkyl” or similar designations. The heteroalkyl group may contain one or more heteroatoms. By way of example only, “C1-4 heteroalkyl” indicates that there are one to four carbon atoms in the heteroalkyl chain and additionally one or more heteroatoms in the backbone of the chain. A heteroalkyl group may be substituted or unsubstituted.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C6-10 aryl,” “C6 or C10 aryl,” or similar designations. For example, the aryl group can be a C6-C14 aryl group, a C6-C10 aryl group, or a C6 aryl group. Examples of aryl groups include, but are not limited to, phenyl, naphthyl, azulenyl, and anthracenyl. An aryl group may be substituted or unsubstituted.

As used herein, “aryloxy” and “arylthio” refers to RO- and RS-, in which R is an aryl as is defined above, such as “C6-10 aryloxy” or “C6-10 arylthio” and the like, including but not limited to phenyloxy. An aryloxy or arylthio group may be substituted or unsubstituted.

An “aralkyl” or “arylalkyl” is an aryl group connected as a substituent via an alkylene group, such “C7-14 aralkyl” and the like, including but not limited to benzyl, 2-phenylethyl, 3-phenylpropyl, and naphthylalkyl. In some cases, the alkylene group is a lower alkylene group (i.e., a C1-6 alkylene group). An aralkyl or arylalkyl group may be substituted or unsubstituted.

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen, and sulfur, in the ring backbone. When the heteroaryl is a ring system, every ring in the system can be aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. For example, the heteroaryl group can contain 4 to 14 ring members (atoms in the ring(s)), 5 to 10 ring members (atoms in the ring(s)), 5 to 7 ring members (atoms in the ring(s)), or 5 to 6 ring members (atoms in the ring(s)). The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furan (e.g., furyl), furazan (e.g., furazanyl), thiophene (e.g., thienyl), benzothiophene (e.g., benzothienyl), phthalazine (e.g., phthalazinyl), pyrrole (e.g., pyrrolyl), oxazole (e.g., oxazolyl), benzoxazole (e.g., benzoxazolyl), 1,2,3-oxadiazole, 1,2,4-oxadiazole, thiazole (e.g., thiazolyl), 1,2,3-thiadiazole, 1,2,4-thiadiazole, benzothiazole (e.g., benzothiazolyl), imidazole (e.g., imidazolyl), benzimidazole (e.g., benzimidazolyl), indole (e.g., indolyl), isoindole (e.g., isoindolyl), indazole, pyrazole (e.g., pyrazolyl), benzopyrazole, isoxazole (e.g., isoxazolyl), benzoisoxazole, isothiazole (e.g., isothiazolyl), triazole (e.g., triazolyl), benzotriazole, thiadiazole (e.g., thiadiazolyl), tetrazole, pyridine (e.g., pyridinyl), pyridazine (e.g., pyridazinyl), pyrimidine (e.g., pyrimidinyl), pyrazine (e.g., pyrazinyl), purine, pteridine, quinoline (e.g., quinolinyl), isoquinoline (e.g., isoquinlinyl), quinazoline, quinoxaline, cinnoline, and triazine (e.g., triazinyl). Heteroaryl rings may also include bridge head nitrogen atoms, for example but not limited to pyrazolo[1,5-a]pyridine, imidazo[1,2-a]pyridine, and pyrazolo[1,5-a]pyrimidine. A heteroaryl group may be substituted or unsubstituted.

A “heteroaralkyl” or “heteroarylalkyl” is a heteroaryl group connected as a substituent via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. A heteroaralkyl group may be substituted or unsubstituted.

As used herein, “carbocyclyl” means a non-aromatic cyclic ring or ring system containing only carbon atoms in the ring system backbone. When the carbocyclyl is a ring system, two or more rings may be joined together in a fused, bridged, or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group may also be a carbocyclyl having 3 to 6 carbon atoms. The carbocyclyl group may be designated as “C3-6 carbocyclyl” or similar designations. Examples of carbocyclyl rings include but are not limited to cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl. A carbocyclyl group may be substituted or unsubstituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups may contain 3 to 10 atoms in the ring(s), 3 to 8 atoms in the ring(s), or as otherwise noted herein. A cycloalkyl group may be unsubstituted or substituted. Typical cycloalkyl groups include but are in no way limited to cyclopropyl, cyclobutyl, cyclopentyl, cyclohexyl, cycloheptyl, and cyclooctyl.

A “(carbocyclyl)alkyl” is a carbocyclyl group connected as a substituent via an alkylene group, such as “C4-10 (carbocyclyl)alkyl” and the like, including but not limited to cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkenyl” means a carbocyclyl ring or ring system having at least one double bond, wherein no ring in the ring system is aromatic. An example is cyclohexenyl. Cycloalkenyl groups can contain 4 to 10 atoms in the ring(s). A cycloalkenyl group may be substituted or unsubstituted.

As used herein, “heterocyclyl” or “heteroalicyclyl” refers to three-, four-, five-, six-, seven-eight-, nine-, ten-, or up to 20-membered monocyclic, bicyclic, or tricyclic ring systems wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) may be present in either a non-aromatic or aromatic ring in the ring system. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged, or spiro-connected fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, or 3,4-methylenedioxyphenyl). The heterocyclyl group may also be a medium size heterocyclyl having 3 to 10 ring members. The heterocyclyl group may also be a heterocyclyl having 3 to 6 ring members. The heterocyclyl group may be designated as “3-6 membered heterocyclyl” or similar designations. A heterocyclyl group may be substituted or unsubstituted.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline. A sulfur of the heterocyclyl ring may be provided as a dioxide (e.g., —S(O)2-).

A “(heterocyclyl)alkyl” is a heterocyclyl group connected, as a substituent, via an alkylene group. Examples include, but are not limited to, imidazolinylmethyl and indolinylethyl. A “(heterocyclyl)alkynyl” is a heterocyclyl group connected, as a substituent, via an alkynylene group.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl. An acyl group may be substituted or unsubstituted.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. An O-carboxy can be substituted or unsubstituted.

A “C-carboxy” group (or “ester”) refers to a “—C(═O)OR” group in which R is selected from hydrogen, —NH₂, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH). A C-carboxy can be substituted or unsubstituted.

As used herein, the term “hydroxy” refers to a —OH group. A “cyano” group refers to a “—CN” group. A “cyanato” group refers to an “—OCN” group. An “isocyanato” group refers to a “—NCO” group. A “thiocyanato” group refers to a “—SCN” group. An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfinyl can be substituted or unsubstituted.

A “sulfonyl” group refers to an “—SO₂R” or “—SO₂-” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. A sulfonyl can be provided in a heterocyclyl ring. A sulfonyl can be substituted or unsubstituted.

A “S-sulfonamido” group refers to a “—SO2NRARB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. RA and RB may be taken together to provide a heteroaryl or heterocycle. A S-sulfonamido can be substituted or unsubstituted.

A “N-sulfonamido” group refers to a “—N(RA)SO₂RB” group in which RA and RB are each independently selected from hydrogen C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-sulfonamido can be substituted or unsubstituted.

An “O-carbamyl” group refers to a “—OC(═O)NRARB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. RA and RB may be taken together to provide a heteroaryl or heterocycle. An O-carbamyl can be substituted or unsubstituted.

An “N-carbamyl” group refers to an “—N(RA)OC(═O)RB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-carbamyl can be substituted or unsubstituted.

An “O-thiocarbamyl” group refers to a “—OC(═S)NRARB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. RA and RB may be taken together to provide a heteroaryl or heterocycle. An O-thiocarbamyl can be substituted or unsubstituted.

A “N-thiocarbamyl” group refers to an “—N(RA)OC(═S)RB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. An N-thiocarbamyl can be substituted or unsubstituted.

A “C-amido” group refers to a “—C(═O)NRARB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, and 5-10 membered heterocyclyl, as defined herein. RA and RB may be taken together to provide a heteroaryl or heterocycle. A C-amido can be substituted or unsubstituted.

A “N-amido” group refers to a “—N(RA)C(═O)RB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl 5-10 membered heteroaryl, and 5-10 membered heterocyclyl, as defined herein. RA and RB may be taken together to provide a heteroaryl or heterocycle. An N-amido can be substituted or unsubstituted.

A “carbamido” or “carbamide” group refers to a “(RARB)NC(═O)N(RC)-” group in which RA, RB, and RC can be independently hydrogen, alkyl, alkenyl, alkynyl, carbocyclyl, aryl, heteroaryl, heterocyclyl, aralkyl, or heterocyclyl(alkyl), as defined herein. A carbamido may be substituted or unsubstituted.

An “amino” group refers to a “—NRARB” group in which RA and RB are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. RA and RB may be taken together to provide a heteroaryl or heterocycle. An amino can be substituted or unsubstituted.

An “alkamino” group refers to a “—NRARB” group in which RA is C₁₋₆ alkylene (e.g., connected to an indicated structural substituent), H, a lone electron pair, or C₁₋₆ alkyl and RB is independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein. Alternatively, an “alkamino” group may be represented as “—N═RB”. An alkamino can be substituted or unsubstituted.

An “aminoalkyl” group refers to an amino group connected via an alkylene group. An aminoalkyl can be substituted or unsubstituted.

An “alkoxyalkyl” group refers to an alkoxy group connected via an alkylene group, such as a “C₂-8 alkoxyalkyl” and the like. An alkoxyalkyl can be substituted or unsubstituted.
As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substituents independently selected from C₁-C₆ alkyl (optionally substituted with —OH, halogen, or C-carboxy), C₁-C₆ alkenyl, C₁-C₆ alkynyl, C₁-C₆ heteroalkyl, C₃-C₇ carbocyclyl (optionally substituted with halo, —OH, C₁-C₆alkyl, C₁-C₆alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with N-amido, —OH, halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), haloalkoxy, cycloalkenyl, halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, a mono-substituted amine group, a di-substituted amine group, a mono-substituted amine(alkyl), a di-substituted amine(alkyl), nitro, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfenyl, sulfinyl, sulfonyl, —O—NH₂, oxo (═O), a diamino-group, a polyamino, a diether-group, and a polyether (e.g., diethylene glycol, triethylene glycol, oligoethylene glycol, polyethylene glycol, etc.). Wherever a group is described as “optionally substituted” (or other similar language) or as comprising one or more “optional substitutions,” that group can be substituted with the above substituents or can be unsubstituted.

In some embodiments, substituted group(s) is (are) substituted with one or more substituent(s) individually and independently selected from C₁-C₄ alkyl, amino, hydroxy, and halogen.

Two substituents may come together with the atom or atoms to which they are attached to form a ring that is spiro or fused with the rest of the compound.

As used herein, any “R” group(s) such as, without limitation, R¹, R², R³, etc., represent substituents that can be attached to the indicated atom. An R group may be substituted or unsubstituted. If two “R” groups are described as being “taken together” (or similar language), the R groups and the atoms they are attached to can form a cycloalkyl, aryl, heteroaryl or heterocycle. When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

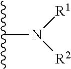

and R¹ and R² are defined as selected from the group consisting of hydrogen and alkyl, or R¹ and R² are defined as selected from the group consisting of hydrogen and alkyl, and R² together with the nitrogen to which they are attached form a heterocyclyl, it is meant that R¹ and R² can be selected from hydrogen or alkyl, or alternatively, the substructure has structure:

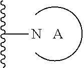

where ring A is a heterocyclyl ring containing the depicted nitrogen. As further illustration, without limitation, if R^{1a} and R^{1b} of an NR^{1a}R^{1b} group are indicated to be “taken together,” it means that they are covalently bonded to one another to form a ring:

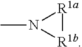

A cyclic structure may be shown using the following structure (or a similar structure with a different ring size, heteroatoms, etc.):

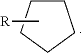

When a cyclic structure is illustrated using this type of structure, what is meant is that the R group may be attached to any position of the ring by replacing an —H of the ring with —R. For example, for a heterocyclic ring includes any of the following ring structures:

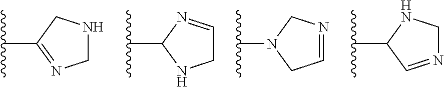

where [symbol] indicates a bond to a remaining portion of the structure. Likewise, the following structure:

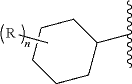

where [symbol] indicates a bond to a remaining portion of the structure and n is 1 to 5, any of the following structures are envisioned or other variations (as would be readily appreciated by the one of skill in the art):

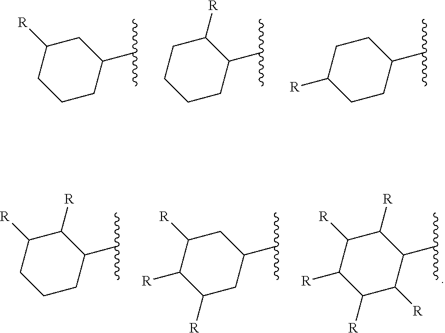

When two “adjacent” R groups are said to form a ring “together with the atoms to which they are attached,” it is meant that the collective unit of the atoms, intervening bonds, and the two R groups are the recited ring. For example, when the following substructure is present:

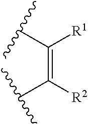

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

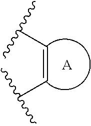

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent A
depicted as -AE- or

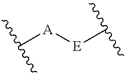

includes the substituent being oriented such that the “A” is attached at the leftmost attachment point of the molecule as well as the case in which “A” is attached at the rightmost attachment point of the molecule.

As noted in the definition for alkylene, it also is to be understood that certain radical naming conventions can include either a mono-radical or a di-radical, depending on the context. For example, where a substituent (e.g., in a genus structure) requires two points of attachment to the rest of the molecule, it is understood that the substituent is a di-radical. For example, a substituent identified as aminoalkyl that requires two points of attachment includes di-radicals such as—NHCH2-, —NHCH₂CH₂—, —NHCH₂CH(CH₃)CH₂—, and the like. Other examples a substituent may require two points of attachment include alkoxy, aryl, heteroaryl, carbocyclyl, heterocyclyl, etc.

As used herein, a radical indicates species with a single, unpaired electron such that the species containing the radical can be covalently bonded to another species. Hence, in this context, a radical is not necessarily a free radical. Rather, a radical indicates a specific portion of a larger molecule.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is understood that, in any compound described herein having one or more chiral centers, all possible diastereomers are also envisioned. It is understood that, in any compound described herein all tautomers are envisioned. It is also understood that, in any compound described herein, all isotopes of the included atoms are envisioned. For example, any instance of hydrogen, may include hydrogen-1 (protium), hydrogen-2 (deuterium), hydrogen-3 (tritium) or other isotopes; any instance of carbon may include carbon-12, carbon-13, carbon-14, or other isotopes; any instance of oxygen may include oxygen-16, oxygen-17, oxygen-18, or other isotopes; any instance of fluorine may include one or more of fluorine-18, fluorine-19, or other isotopes; any instance of sulfur may include one or more of sulfur-32, sulfur-34, sulfur-35, sulfur-36, or other isotopes.

In several embodiments, where an intermediate variable (e.g., a variable that is between two other structural features) is described as optionally being “not present,” when not present the variable can be expressed as a single bond between two adjacent groups. Alternatively, that intermediate variable may be defined as a single bond.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps but may include additional steps. When used in the context of a compound, composition or device, the term “comprising” means that the compound, composition or device includes at least the recited features or components but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

### Compounds

Several embodiments disclosed herein are a compound or composition with the structure below:

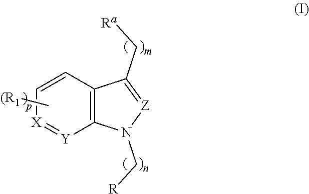

where X, Y, and Z are independently either N or CH; R optionally substituted alkyl, optionally substituted cycloalkyl, optionally substituted heteroalkyl, optionally substituted heterocyclyl, optionally substituted aryl, optionally substituted heteroaryl, optionally substituted carbamido, optionally substituted alkamino, optionally substituted sulfonyl, and optionally substituted N-sulfonamido; n is 0, 1, 2, or 3; R1 is selected from a group consisting of optionally substituted alkyl, halogen, optionally substituted alkoxy, and a hydrogen isotope; p is 0, 1, 2, 3, or 4; Ra is selected from a group consisting of hydroxyl, optionally substituted alkyl, optionally substituted cycloalkyl, optionally substituted aryl, optionally substituted heteroaryl, and optionally substituted heteroalkyl; and m is 0, 1, 2, or 3.

In several embodiments, when n is 1, and Ra is OH, n is not 1. In several embodiments, when m is 1, Ra is OH, and n is 1, then R is not aryl. In several embodiments, when m is 1, Ra is OH, n is 1, and R is aryl, then R is not substituted with —OMe.

In several embodiments, where substituted, each substitution of R is selected from a group consisting of a hydrogen isotope, —OH, —NH2, —CN, halogen C1-6 alkyl, C1-6 alkoxy, C1-6 alkyl substituted with hydroxyl or amino, C1-6 alkamino, ═O, ═NH, CF3, OCF3, —CH2OH, COOH, CHO, and phenyl, or, where two substitutions are present, they come together to provide a cyclyl group or heterocyclyl group.

In several embodiments, R₁ is selected from the group consisting of optionally substituted C₁₋₆ alkyl and optionally substituted C₁₋₆ alkoxy. In several embodiments, R₁ is selected from the group consisting of —CH₃, —OCH₃, —CF₃, and —OCF₃.

In several embodiments, where substituted, each substitution of Ra is selected from a group consisting of a hydrogen isotope, —OH, —NH₂, —CN, halogen, C₁₋₆ alkyl, C₁₋₆ alkoxy, C₁₋₆ alkyl substituted with hydroxyl or amino, C₁₋₆ alkamino, ═O, ═NH, CF₃, OCF₃, —CH₂OH, COOH, CHO, and phenyl, or, where two substitutions are present, they come together to provide a cyclyl group or heterocyclyl group.

In several embodiments, where R or Ra comprises a heteroatom, the heteroatom is selected from S, O, and N.

In several embodiments, Ra is hydroxyl. In several embodiments, wherein m is 1.

In several embodiments, n is 0 or 1. In several embodiments, p is 0.

In several embodiments, X is CH. In several embodiments, Y is CH. In several embodiments, Z is CH.

In several embodiments, R is selected from a group consisting of optionally substituted phenyl, thiophene, pyridine, pyrrole, furan, morpholine, piperidine, pyrroline, pyrazole, pyrimidine, pyrrolidine, pyridazine, imidazole, imidazolidine, and isoxazole; and wherein the one or more substitutions are selected from a group consisting of CH₃, OCH₃, F, OH, NH₂, CH₂NH₂, ═O, CH₂OH, CH₂CH₃, CN, ═NH, and morpholine. In several embodiments, R is NH(CO)NH-phenyl.

In several embodiments, R is N-cyclohexane. In several embodiments, R is —NHS(O)2-benzene. In several embodiments, R is N(CH₃)S(O)2-benzene. In several embodiments, R is S(O)2-benzene. In several embodiments, R is molecule-cg19. In several embodiments, R is N=cyclopentane. In several embodiments, R is a cyclic structure with a 1C- or 2C-bridge.

### 1. Generating N-Substituted Indole Carbinols

Several embodiments pertain to a compound of Formula (Ia) (and including deuterated and other isotopes of the R groups):

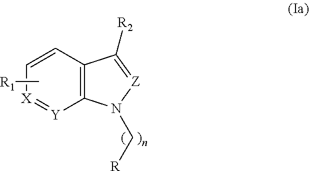

wherein X, Y and Z are independently N or CH; R is substituted aryl, alkyl, branched alkyl, substituted cycloalkyl, substituted heteroaryl, substituted heteroalkyl, wherein the substitutions include alkyl, halogens, hydrogen isotopes, and substitutions yielding bridged bicyclic compounds; n is 0-3; R¹ is alkyl, branched alkyl, substituted alkyl, substituted branched alkyl, halogen, and hydrogen isotopes, wherein substitutions include but not limited to —CF₃, —OCF₃, and —CH₂OH; and R₂ is substituted or unsubstituted alkyl, branched alkyl, cycloalkyls, heterocycloalkyl, bicyclic heterocycloalkyl, and heteroaryl, where substitutions include S, O, N, —CH₂OH, COOH, and CHO.

### Compositions

Several embodiments pertain to compositions comprising compounds as disclosed herein. In several embodiments, the cyclodextrin is a β-cyclodextrin or a γ-cyclodextrin. In several embodiments, the β-cyclodextrin is methyl-β-cyclodextrin. In several embodiments, the β-cyclodextrin is Captisol®. In several embodiments, the composition comprises a carrier.

In several embodiments, a liquid pharmaceutical composition is used. In several embodiments, a liquid pharmaceutical composition, as used herein, whether in the form of a solution, suspension or other like form, may include one or more of the following carriers or excipients: sterile diluents such as water for injection, saline solution, preferably physiological saline, Ringer's solution, isotonic sodium chloride, fixed oils such as squalene, squalane, mineral oil, a mannide monooleate, cholesterol, and/or synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. In several embodiments, the pharmaceutical composition is sterile.

### Methods of Use

Several embodiments pertain to a method of treating a disease in a subject in need thereof. In several embodiments, the method comprising administering to said subject a composition or compound as disclosed herein. In several embodiments, the disease is a cancer. In several embodiments, the cancer is selected from a group consisting of breast cancer, lung cancer, prostate cancer, kidney cancer, pancreatic cancer, liver cancer, brain cancer, ovarian cancer, colon cancer, leukemia, lymphoma, multiple myeloma, thyroid cancer, bone cancer, and esophageal cancer. In several embodiments, the compound is administered parenterally, orally, subcutaneously, topically, rectally, intrathecally, intracerebrally, intramuscularly, epidurally, intravenously, intraperitoneally, vaginally, or pulmonarily.

In several embodiments, the pharmaceutical composition is administered in a single dose or at multiple times or is delivered substantially uniformly over a period of time during which the dose of the compound can be varied over time.

Several embodiments pertain to a method to identify subjects that can benefit from treatment with a compound or composition as disclosed herein, the method comprising determining the level of SULT1A1 mRNA or protein expression and selecting patients with expression above a threshold. In several embodiments, the method of determining the level of SULT1A1 expression comprises at least one of protein detection by immunohistochemistry, Western Blot, or enzyme-linked immunosorbent assay or RNA detection by RNA sequencing, qRT-PCR, or RNA microarray.

### Methods of Manufacture and Design

Several embodiments pertain to methods of making compounds as disclosed elsewhere herein. Several embodiments pertain to the design of compounds disclosed herein. In several embodiments, the compound comprises an indole ring. In several embodiments, the compound comprises an electron withdrawing group on the phenyl ring of the indole. In several embodiments, the compound comprises an electron donating group on the phenyl ring of the indole. Exemplary compounds are shown below:

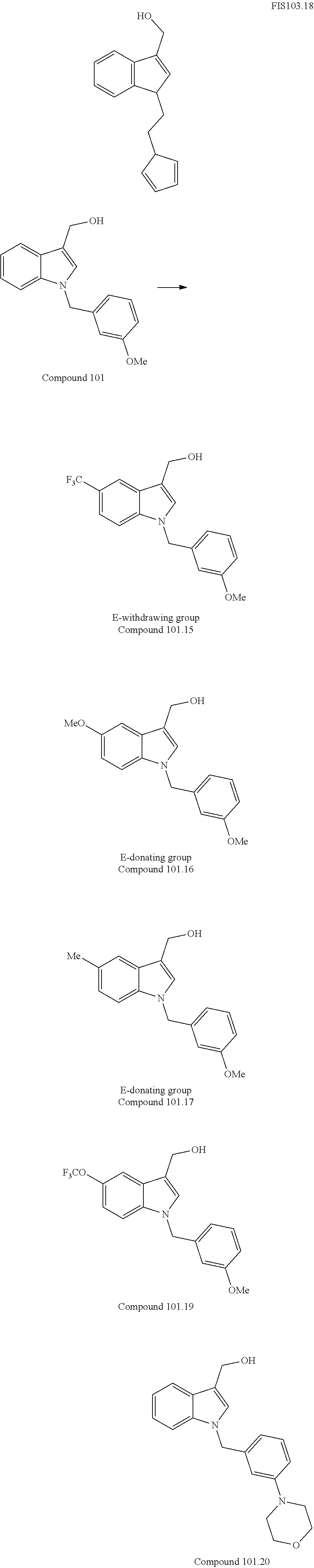

### EXAMPLES

### Example 1A. Compound 103 and its Efficacy In Vitro and In Vivo

Following screening of multiple analogs in silico, selected analogs were synthesized and evaluated for efficacy and stability. Among the analogs generated, Compound 103 (shown above in the Summary) demonstrated enhanced stability, while maintaining SULT1A1-dependent antitumor potency.

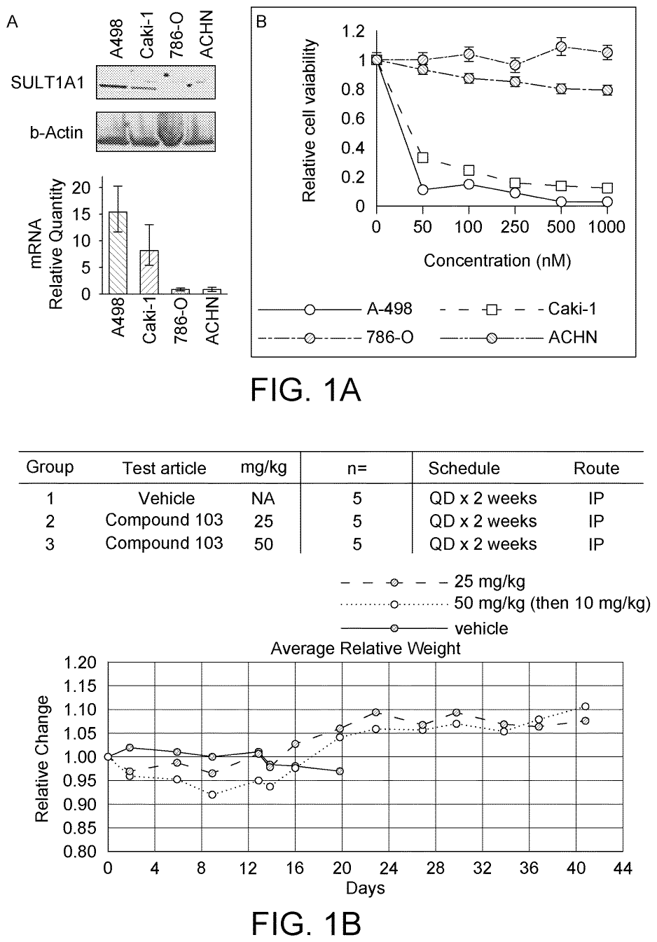

Compound 103's antitumor activity was evaluated in 4 RCC cell lines whose SULT1A1 expression is either high (A498 and Caki-1) or low (786-0 and ACHN). The result clearly demonstrated Compound 103 potency in SULT1A1 highly expressing cells but not in cells with low SULT1A1 expression (FIG. 1A). As A498 is a primary kidney carcinoma cell line and Caki-1 is a metastatic (skin) kidney carcinoma cell line, this result suggests Compound 103 can be efficacious in both primary and metastatic tumors expressing SULT1A1.

While Compound 103 was more stable than other analogs generated, and was insoluble in aqueous solvents, degradation was still observed. A physiologically relevant and preferred aqueous formulation was found to be a composition of 15 mM Compound 103 and 50 mg/ml of 2-hydroxypropyl-beta-cyclodextrin (HPβCD), and in this composition, Compound 103 was completely solubilized and stable at 2-8 °C or at room temperature for over one month (See FIG. 11).

The efficacy of the preferred formulation (15 mM Compound 103 and 50 mg/ml HPβCD) was tested in vivo using a A498 subcutaneous xenograft model. Once the tumors reached ~150 mm³, vehicle control or Compound 103 (25 mg/kg) were injected IP daily for 14 days. As early as day 3, tumor regression (based on average animal weight gain) was noticed in the treated group (FIG. 1B) with complete regression by ~Day 6. Mice were observed for an additional 16 days post treatment. While tumors in the control mice continued to grow (based on average animal weight gain) until animals had to be sacrificed, those in the treated group remained undetectable with no sign of recurrence. All Compound 103-treated mice were healthy and behaved normally throughout the study, suggesting a favorable toxicity profile of Compound 103.

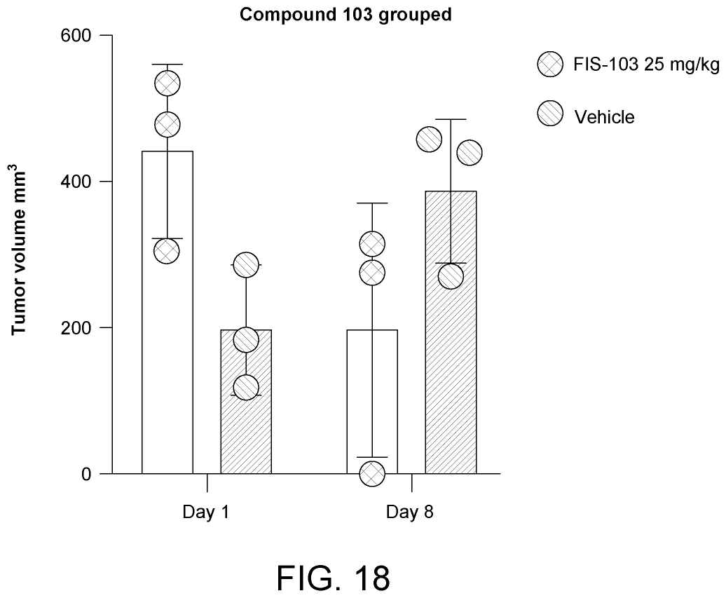

In FIG. 18, A498 renal carcinoma cells, a SULT1A1-positive tumor model, were implanted into Nu/J mice (Jax labs). Once tumors reached approximately 75 mm³, mice were treated with Compound 103 (25 mg/kg, intraperitoneally) for four consecutive days. The results show that after tumor growth was monitored through day 8 of a study (n=3 mice per group, represented by dots in the bars) treatment with Compound 103 resulted in a marked reduction in tumor volume compared to vehicle control, and one treated mouse (at 0 on the Y axis) had no measurable tumor by day 8—at which point the animal was terminated from the study).

### Example 1B. Sulfonation Assay for Compound 103

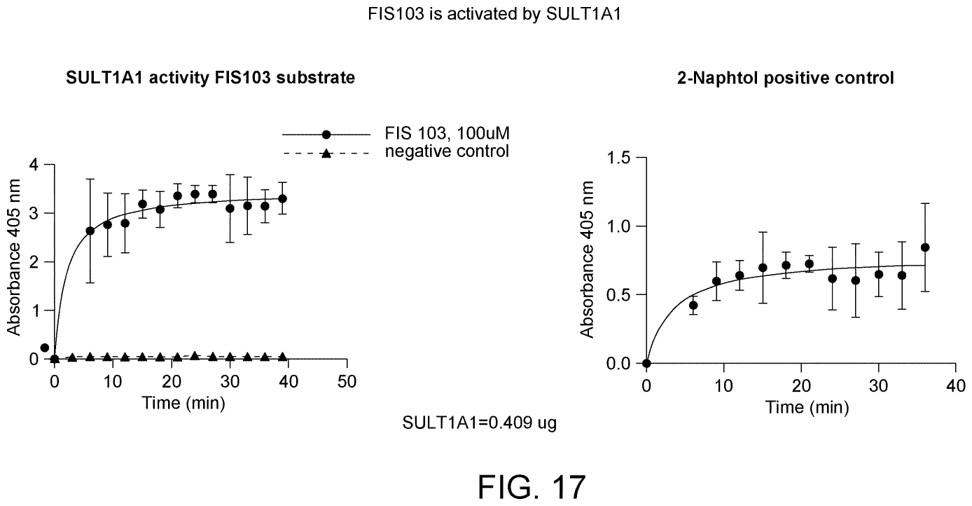

FIG. 17 depicts results from a sulfonation assay, showing that Compound 103 (0.5 mM) is sulfonylated by SULT1A1 in the presence of p-nitrophenyl sulfate (sulfonate donor) and 5′-phosphoadenosine-3′-phosphosulfate (PAPS), the cofactor used by sulfotransferases such as SULT1A1 to transfer a sulfate group to the substrate. Reactions were performed in duplicate, and the error bars represent S.E.M.

The results indicate that Compound 103 is a prodrug that is enzymatically activated by SULT1A1, converting it into a potent electrophilic alkylating agent that induces apoptosis in tumor cells.

### Example 2. Additional Computational Chemistry to Generate Additional Analogs

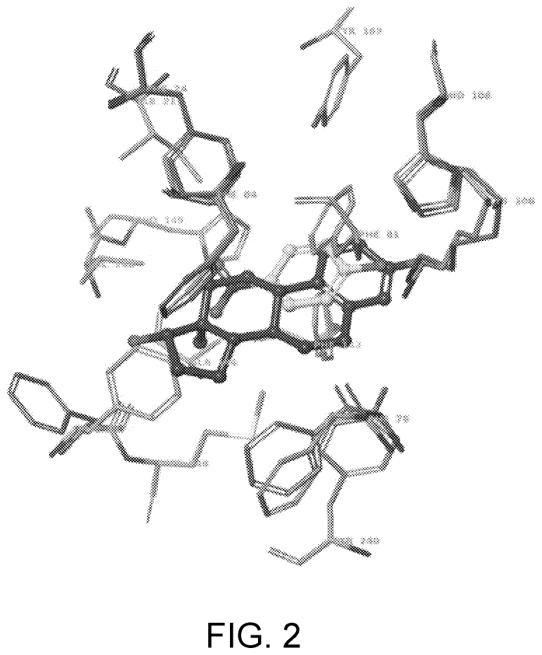

The success of Compound 103 encouraged identifying additional analogs to create a pool of compounds for development. The discovery of novel N-substituted Indole Carbinols started with identifying the crystal structure of the SULT1A1 protein. A literature search revealed that there are a number of good resolution SULT1A1 crystal structures available (PDB IDs: 2D06, 1LS6, 3QVU, 3QVV, 3U3K, 3U3M, 3U30 etc). These PDB IDs were superimposed and the conformational differences at substrate binding sites among different SULT1A1 crystal structures were analyzed. It was found that these structures broadly fall under two cluster of conformation and PDB IDs 2D06 and 1LS6 are the representative members of their cluster. FIG. 2 shows the aligned and superimposed structures of 2D06 and 1LS6. The substrate binding site of SULT1A1 comprises mainly a bulk hydrophobic cavity made up by Phe-142, Phe247, Phe24, Phe84, and Phe76. However, Lys106 and His108 also participate in ligand interactions at one end. Comparisons between PDB IDs 2D06 and 1LS6 suggest that Tyr240, Phe76 and Phe247 residues are displaced in 2D06 because of the larger molecular size of estradiol; hence both structures were selected for structure based virtual screening.

### Example 3. Docking Studies, Binding Site Model for Compound 103

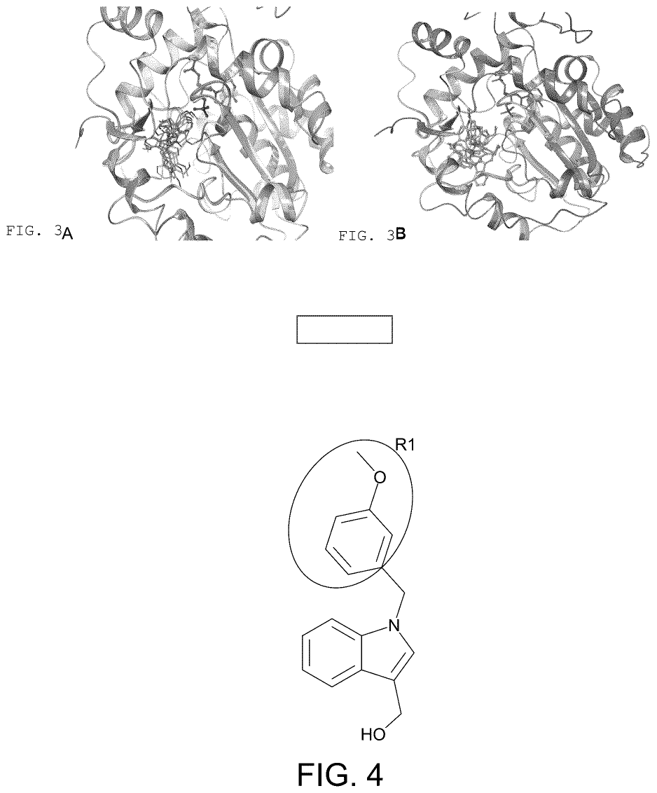

Compound 103 was docked at the defined grid (using the co-crystallized ligands) using the automated “Standard precision” (SP) mode of Glide to check if the binding mode of Compound 103 at the SULT1A1 binding site is similar to p-nitrophenol or estradiol. The docking outputs does suggest that Compound 103 binding at the SULT1A1 protein is very similar to the co-crystallized p-nitrophenol or estradiol binding. To further confirm this the top scoring docking model for 2D06 and 1LS6 were submitted to a short 100 ns MD simulations using Gromacs v2020. The trajectories were well converged as assessed by the time evolution of backbone atoms root mean square deviation (RMSD) from the starting structure. The ligand RMSD analysis from the starting Compound 103 structure from MD simulation was inconclusive because the ligands kept reorienting themselves at the binding site during the simulation. Hence, for the docking and post docking analysis we picked the lowest energy conformation of Compound 103 which aligns with co-crystallized conformation of estradiol and p-nitrophenol with 2D06 and 1LS6. FIGS. 3A and 3B show the cluster representative structure of Compound 103 during 100 ns MD simulation with 2D06 (shown in FIG. 3A) and 1LS6 (shown in FIG. 3B).

### Tailoring Ligand Libraries:

The fragment-based method for the generation of a novel and focused virtual library was used in the present study using the Custom R-group Enumeration tool of Schrodinger software which allows the user to run fragment-based generation of a virtual library in a user-friendly interface. Compound 101 “protocore” (FIG. 4) as an indole-3-carbinol, was considered as a starting structure for the library generation using the default parameter settings of the Custom R-group Enumeration tool.

Various pre-defined fragment sets were proposed for high affinity binding, illustrating the suitability of different structural motifs, in terms of different hydrophobic (aromatic/aliphatic), ionizable (positive/negative), and vector (donor/acceptor) features, at R1 positions relative to central protocore. The addition of virtually prepared reagents at selective attachment point “R1”, (FIG. 4) resulted in around 12000 new Compound 103 analogues and homologues.

### Preparation of Proteins and Ligands and Ensemble Docking:

The representative protein structures were prepared for docking using the protein preparation wizard in Schrödinger with default options used to assign bond order, add hydrogen atoms, and remove water molecules 5 Å away from hetero groups. Hydrogen bond assignment was done using PROPK at pH 7.0, and the orientation of hydroxyl groups, side chain amide groups of Asn and Gln, and charge state of His residues were optimized, and the protein was minimized to an RMSD limit from the starting structure of 0.3 Å using the OPLS3 force field. For docking, the x, y, and z coordinates for the center of the grid box were chosen by picking the center of co-crystallized ligands (p-Nitrophenol and estradiol for 1LS6 and 2D06 respectively) and the dimension of the grid box was fixed at 30, 30, and 30 Å in the x, y, and z directions. Ligand libraries were prepared using LigPrep3.4 with Epik3.2 at pH 7.0. Ligand libraries were docked and tailored to each of the 4 representative SA1 proteins using the standard precision (SP) mode of Glide v9.0 of Schrödinger. Chemical diversity was calculated by generating daylight invariant atom type fingerprints followed by clustering using the linkage method implemented in Schrodinger.

### Post-Docking Analysis:

Extensive docking analysis was conducted using the generated Compound 103 analog library containing ~12K ligands, targeted onto two representative structures of SULT1A1. To prioritize predicted hits, the top 5% scoring hits were subjected to hierarchical post-docking analysis, as follows.
1. Select ligands which show indole-3-carbinol core consistent with Compound 103.
2. Docking score <−7 K Cal/mol
3. Remove redundant molecules if any obtained from 1LS6 and 2D06
4. Chemical diversity cluster

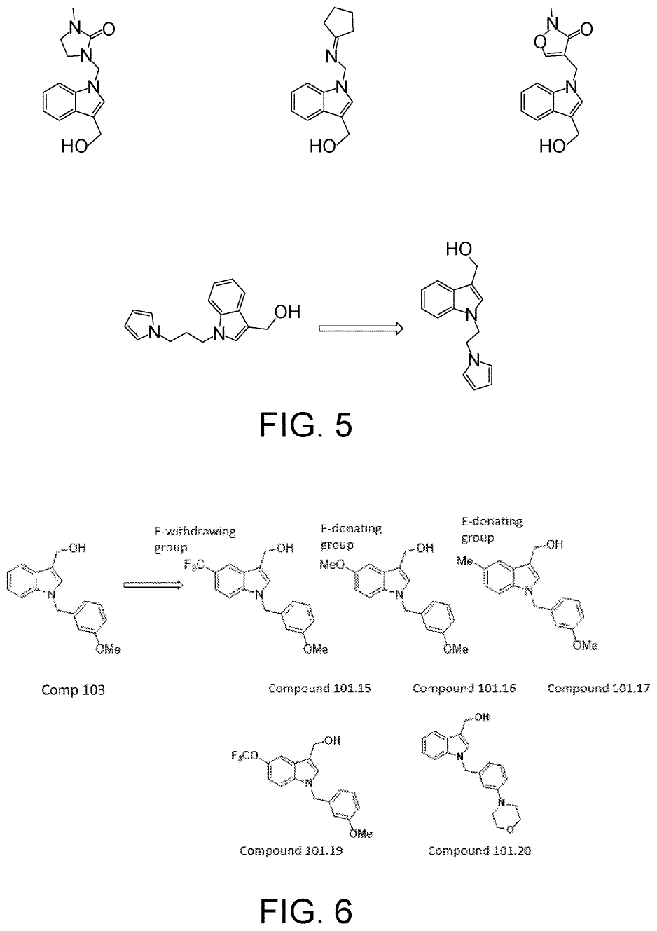

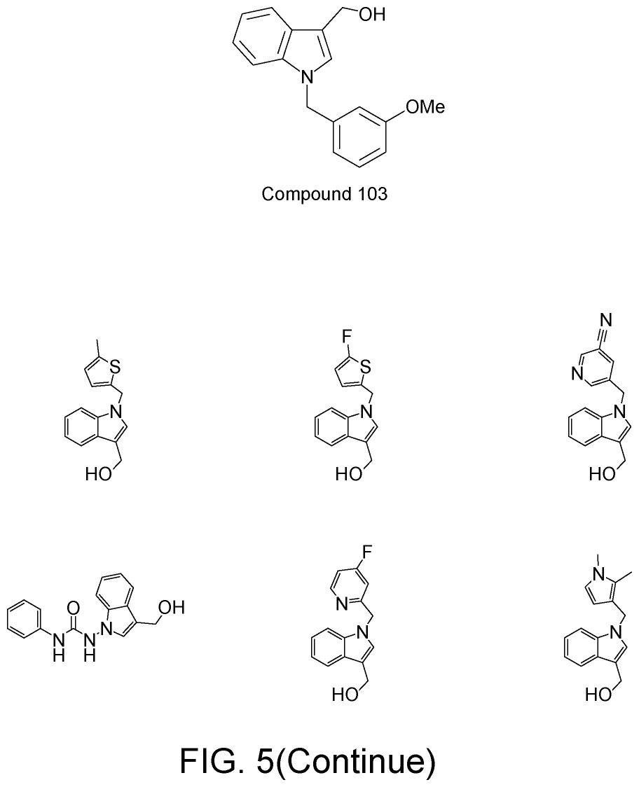

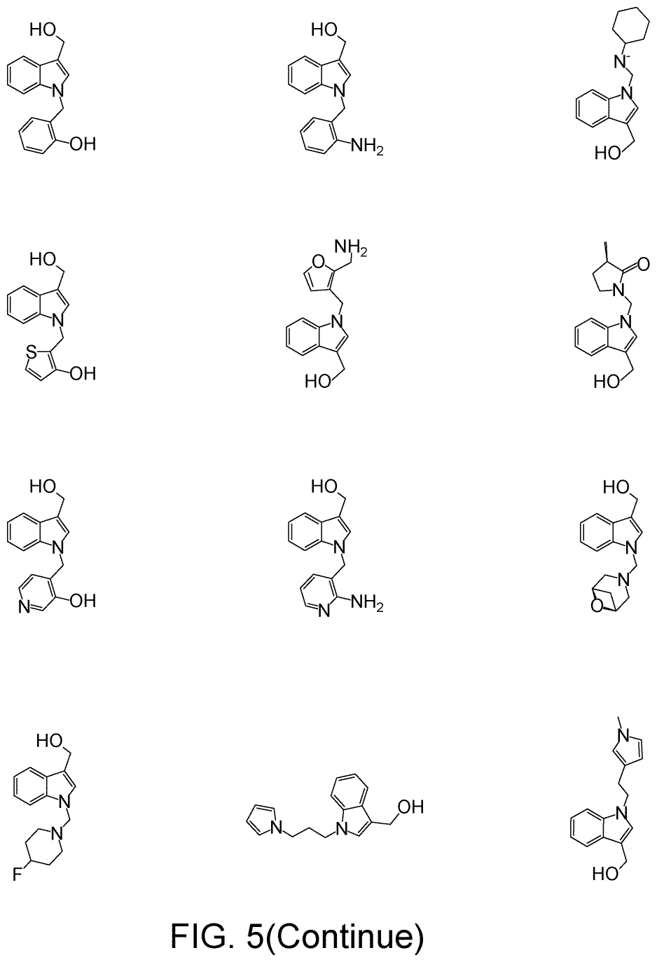

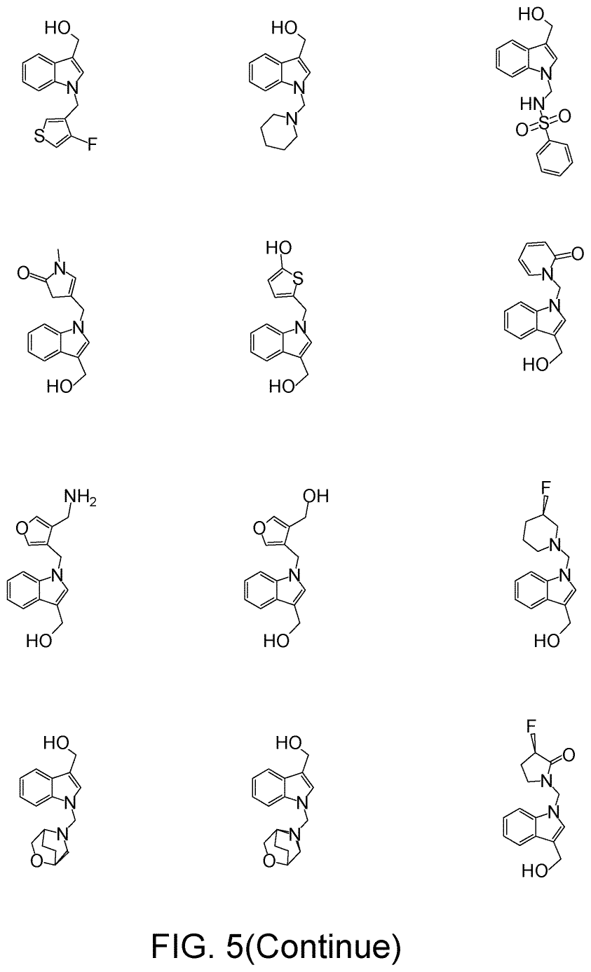

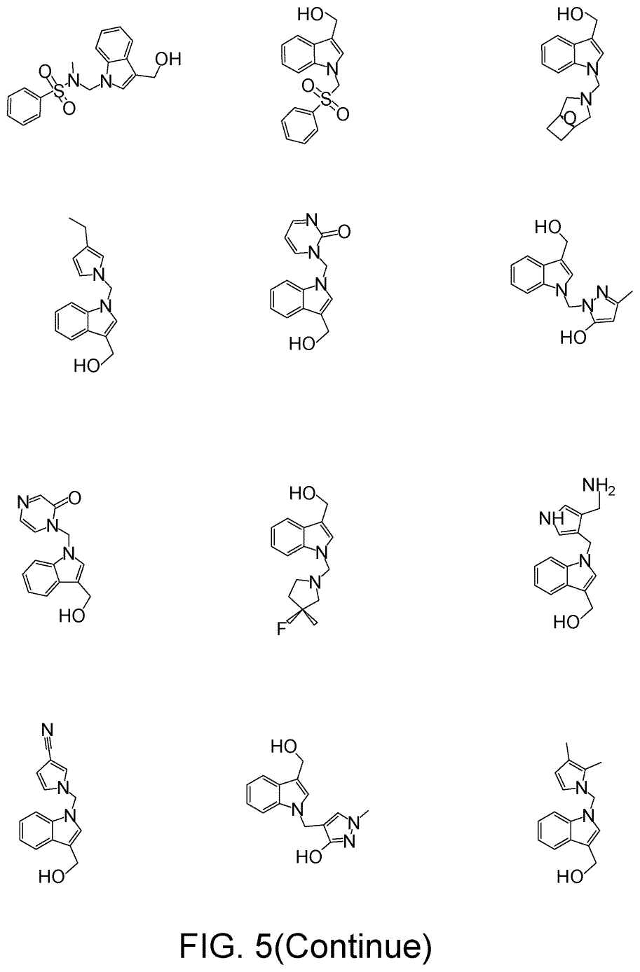

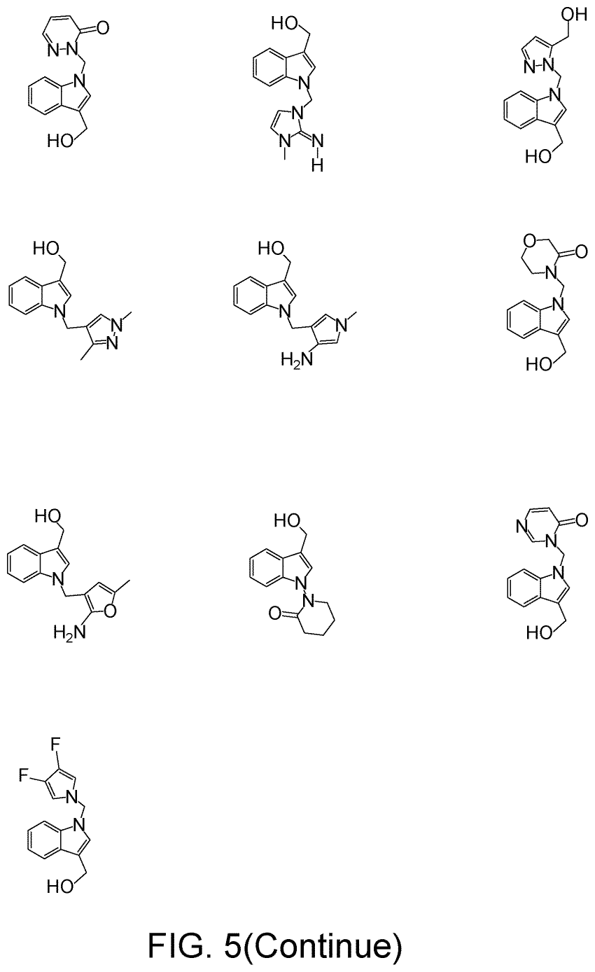

This process led to a list of 82 structurally diverse virtual hits for SULT1A1. These 82 Compound 103 analogs were scrutinized for their novelty (95% or less identity with the known compounds) which gave a 58 novel Compound 103-analogs (FIG. 5).

### Example 4. Medicinal Chemistry

The traditional medicinal chemistry approach was also used to generate analogs. It relied on the availability of a chemical building block, the addition of an electron donating group or an electron withdrawing group, and the use of different linker lengths to increase the bulkiness of the core structure to enhance specificity. This method generated six analog compounds (FIG. 6).

### The Method of Synthesizing the Analogs

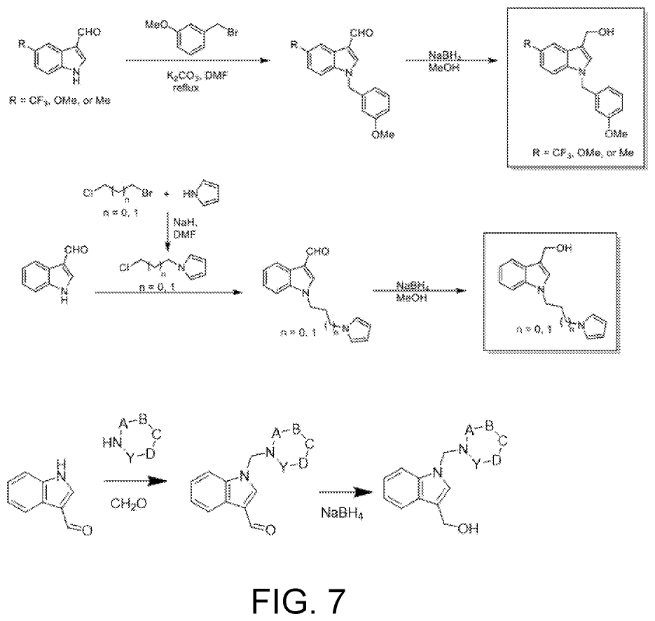

The routes of synthesis for 7 analogs from the 58 analogs of primary interest is shown in FIG. 7. Other compounds of interest and related compounds can be generated by such methods or other well-known methods of synthesis.

### Example 5. Stable Pharmaceutical Formulations for the Compounds and Analogs

Cyclodextrins (CDs) are cyclic oligomers of glucose. CDs have a large number of hydroxyl groups and therefore are generally water soluble. CDs can form water-soluble inclusion complexes with small molecules.

CDs are classified based on the number of glucose residues in their structure. Alpha-CD is glucose hexamer, β-CD is glucose heptamer, and γ-CD is a glucose octomer. Multiple derivatives of CDs have been generated by substituting the CD hydrogen atoms with other molecules such as hydroxypropyl, methyl, and sulfobutylether. Different types of CDs stabilize and/or solubilize compounds differently. Cyclodextrins suitable for stabilizing and/or solubilizing Compound 103 and other analogs include hydroxypropyl-γ-cyclodextrin (HPγCD), methyl-β-cyclodextrin (MβCD), hydroxypropyl-β-cyclodextrin (HPβCD), and sulfobutylether-β-cyclodextrin (SBEβCD).

Table 1 lists the estimated solubility for Compound 103 in various CD solutions at both high (450 mg/mL) and lower (225 mg/mL) CD concentration, and the amount (Amt) of CD solution (in μl) required to visibly dissolve 5 mg of Compound 103.

TABLE 1
Compound 103 estimated solubility in CD solution

| | | | mM | mM |
|---|---|---|---|---|
| FIS | | Amt | (w/ 450 | (w/ 225 |
| Amount (mg) | CD | (μL) | mg/mL CD) | mg/mL CD) |
| 5 | HPγCD | 600 | 31.2* | 10.4 |
| 5 | HPβCD | 700 | 26.7 | 15.6 |
| 5 | SBME | 800 | 23.4 | 18.7 |
| 5 | MβCD | 1100 | 17.0 | 23.4 |

Stability of the CD-Compound 103 solutions was evaluated at 2-8 °C and room temperature (approximately 25 °C). Compound 103 was tested dry, in water, and in phosphate buffered saline (PBS). In the absence of CDs, 30% acetonitrile was used to solubilize Compound 103 in aqueous systems for analysis. Compound 103 was quantified using an Agilent 6140 liquid chromatography mass spectrometry (LC/MS) single quad system with a Kromasil C₁₈ column (5 m, 100 Å, 30×4.60 mm). The detection of Compound 103 at time 0 (TO) for each experiment was used to establish the 100% mark. Subsequent time points were then compared to the TO value to establish the percent recovered.

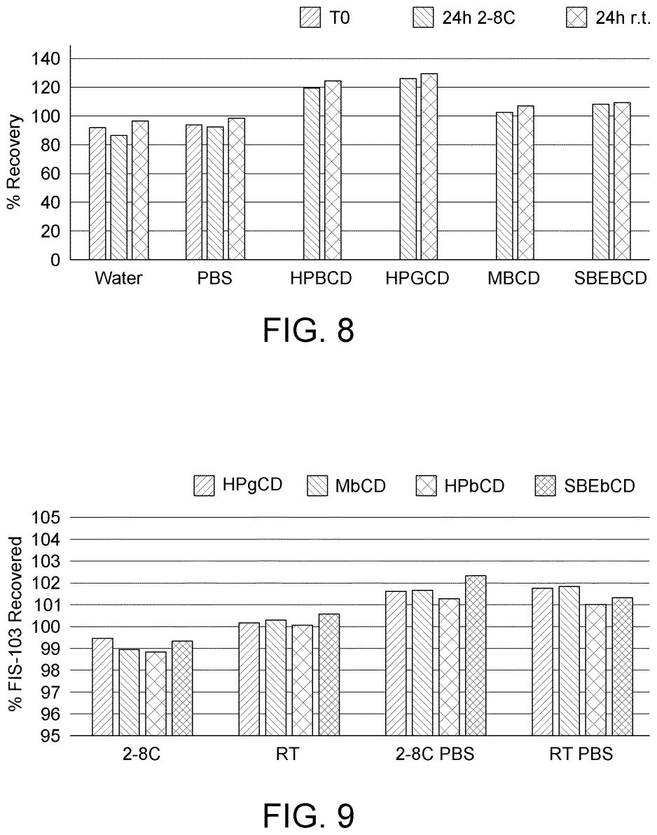

Initial stability tests are summarized in FIG. 8. The stability of neat Compound 103 is good at both 2-8 °C and room temperature. A very important observation with the CDs is that they facilitate the dissolution of Compound 103 into an aqueous system and maintain the stability of the compound. The observation of >100% recovery may be due to continued dissolution of Compound 103 that was not captured at TO. The physical appearance of the Compound 103 gel makes it difficult to visually confirm complete dissolution. While the solutions are given 2 hrs to dissolve and appear clear, small amounts of undissolved Compound 103 may not be visible.

Data for the repeatability of the Compound 103 stability assay are listed in Table 2. The data is consistent and indicative of a stable system. Single time points were used in subsequent tests for efficiency with screening.

TABLE 2
Repeatability of Compound 103 (12.9 mM)
measurements in 310 mg/mL CD solutions

| | Water | | PBS | |
|---|---|---|---|---|
| | 2-8 C | RT | 2-8 C | RT |
| HPγCD | 99.50 ± 0.41 | 100.24 ± 0.78 | 101.65 ± 0.15 | 101.81 ± 0.52 |
| MβCD | 98.97 ± 0.25 | 100.34 ± 0.24 | 101.71 ± 0.74 | 101.86 ± 0.20 |
| HPβCD | 98.87 ± 1.31 | 100.10 ± 0.46 | 101.32 ± 0.59 | 101.06 ± 1.37 |
| SBEβCD | 99.34 ± 1.68 | 100.57 ± 0.25 | 102.37 ± 0.71 | 101.36 ± 0.63 |

Stability tests with all four CDs were expanded to include testing at different temperatures and in PBS solution. The results, as seen in FIG. 9, show continued strong stability at both 2-8 °C and room temperature for 24 hours. The graph Y-axis is narrowed to emphasize the small differences in recovery.

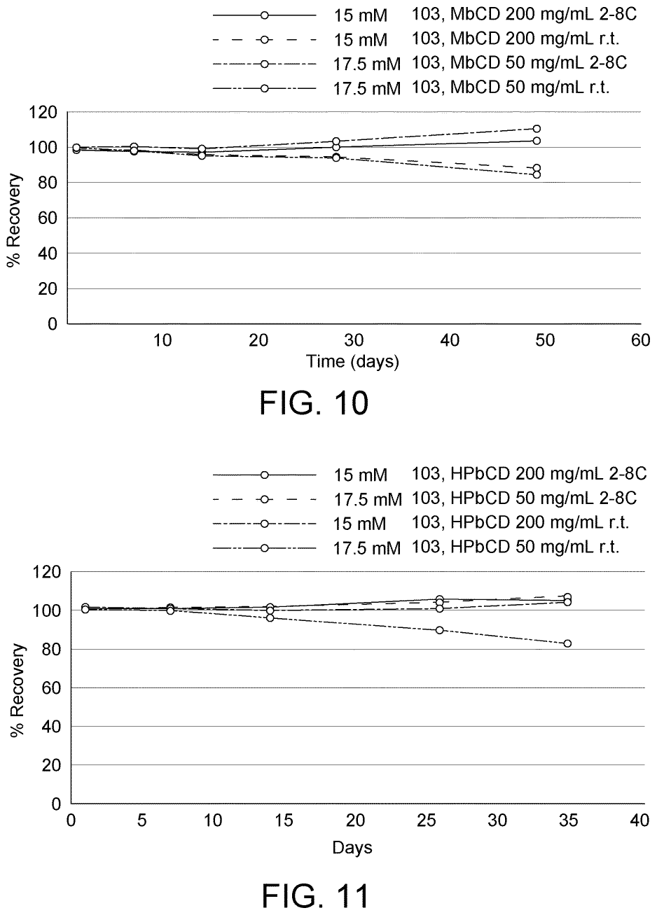

Stability studies of Compound 103 were carried out at 15 mM and 17.5 mM concentrations in 50 and 200 mg/mL PBS solutions of both MβCD (methyl-β-cyclodextrin) and HPβCD (hydroxypropyl-β-cyclodextrin). The tests were performed at 2-8 °C and room temperature for 5 weeks with HPβCD and 7 weeks with MβCD. The results for MβCD are plotted in FIG. 10. A 12-16% loss is observed after 7 weeks for the two samples at room temperature, while the two samples at 2-8 °C show an increase in recovery. The increase is likely due to slow dissolution of the Compound 103 that was not captured in the TO measurement. Therefore, some degradation is likely, but it is obscured by the latent active pharmaceutical ingredient (API) dissolution.

The stability results for Compound 103 in HPβCD solutions are plotted in FIG. 11. Similar trends are observed as with the MβCD solutions (FIG. 10) except: (1) the decrease for the 17.5 mM solution at room temperature is slightly greater, with a decrease of 17% recovered API, and (2) the 15 mM solution does not show a decrease, but rather an increase. This again is likely due to latent dissolution of the Compound 103 unaccounted for at the initial time point.

Compound 103 was successfully formulated into aqueous solutions using modified cyclodextrins. Four modified CDs were screened for use in the solutions, including HPγCD, HPβCD, SBEβCD, and MβCD. The CD solutions were able to dissolve up to 30 mM Compound 103. Samples of Compound 103 show negligible API loss after 7 weeks at room temperature when mixed with MβCD or HPβCD (results not shown). The concentration is also sufficiently high to maintain feasibility of IV administration of a 100 mg/kg dose.

Solutol HS15 (polyethylene glycol (15)-hydroxystearate) can also solubilize and stabilize NSC 743380, which is an N-BIC, i.e., 1-[(3-chlorophenyl)-methyl]-1H-indole-3-carbinol), for more than 4 days.

### Example 6. Testing of Anti-Tumor Effects

### A. In Vitro Efficacy Studies for MβCD Formulated Compound 103

Although Compound 103 is tissue agnostic, for consistency among efficacy studies, a single cancer type, renal cell carcinoma (RCC), was selected. About 9% of RCC patients have SULT1A1 overexpression (Table 1), but the presence of SULT1A1 expression in kidney tubules also makes it a challenging cancer for targeting in vivo. It therefore presents a challenge to Compound 103 activity and a suitable test system.

Compound 103 antitumor activity was evaluated in 4 RCC cell lines whose SULT1A1 expression is either high (A498 and Caki-1) or low (786-0 and ACHN) (FIG. 1A, two left-most panels). Cell viability was measured by MTT after 72 hr of treatment. The results clearly demonstrated Compound 103 potency in SULT1A1 highly expressing cells and lack thereof in cells with low expression (FIG. 1B). Compound 103 was effective without regard to the cell being from a primary cancer (A498) or metastasis (Caki-1).

### B. In Vivo Studies for Compound 103

Evaluation of anti-tumor efficacy of Compound 103 (using a composition of Compound 103/MβCD) was done in an athymic RCC mouse model. Briefly, ~106 cells/mouse were subcutaneously injected into the right hind flank of 6-week old female homozygous NU/J mice. Tumors were measured in two dimensions with a digital caliper for the duration of the study. Tumor volume was calculated using the formula: Tumor Volume (mm³)=0.5×length×width²; where w=width and l=length, in mm, of the tumor. This study consisted of a single efficacy study with 3 total arms of (5 mice) per arm (FIG. 1B—table). On the 1st day of treatment, the mice had tumors ranging from −93-278 mm3. All treatments were administered by intraperitoneal injection (IP). After two days of dosing, it was noticed that the 50 mg/kg treated mice were showing signs that the drug was not being tolerated, and 2 mice died in the study. Treatment in this group was stopped for three days (washout), then the dose was reduced to 10 mg/kg.

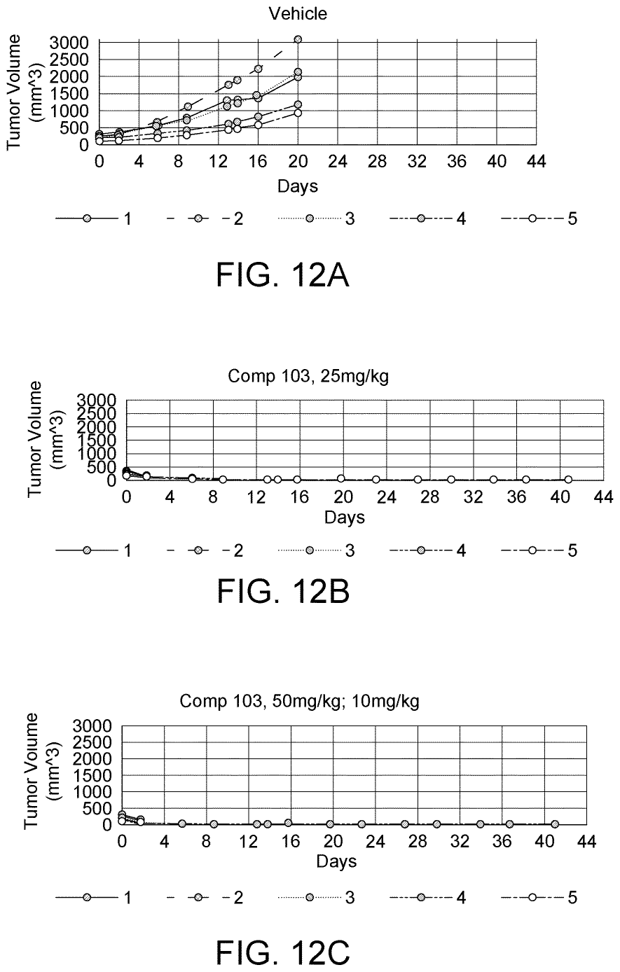

The mice in the vehicle control group exhibited stable weight at the beginning of the study followed by a slow decrease in weight (FIG. 1B—graph), likely attributed to growing tumor burden. The 25 mg/kg group lost about 4% body weight for a few days and then fully recovered. The 50 mg/kg group lost nearly 10% of body weight, but fully recovered after the drug holiday and change of dose to 10 mg/kg. Initially, general loss of vigor and weight loss were noted for Compound 103 treated mice, but both 10 and 25 mg/kg groups appeared to tolerate the drug and dosing schedule well. Tumors in the control group grew for 20 days (FIG. 12A) before the mice were sacrificed due to excess tumor burden. All mice in the Compound 103 treatment groups (FIGS. 12B, 12C) had tumors that became non-detectable after 14 days and remained absent though day 41 (study conclusion).

Dosage and regimens for human treatment with the compositions herein can be determined by extrapolation, based on weight difference from mice.

### Example 7. Testing of BST-10 and BST-12

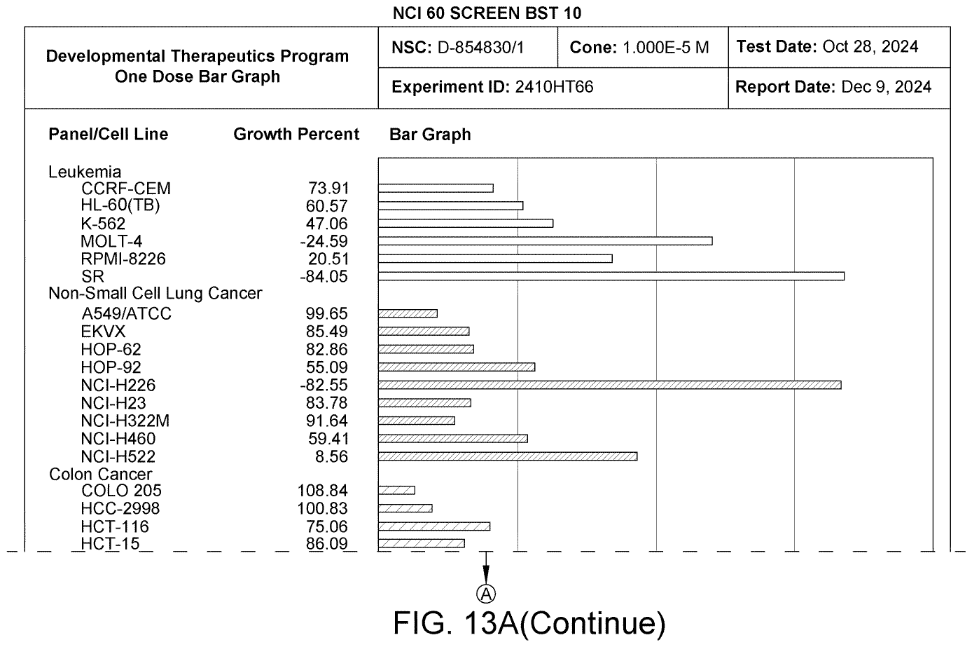

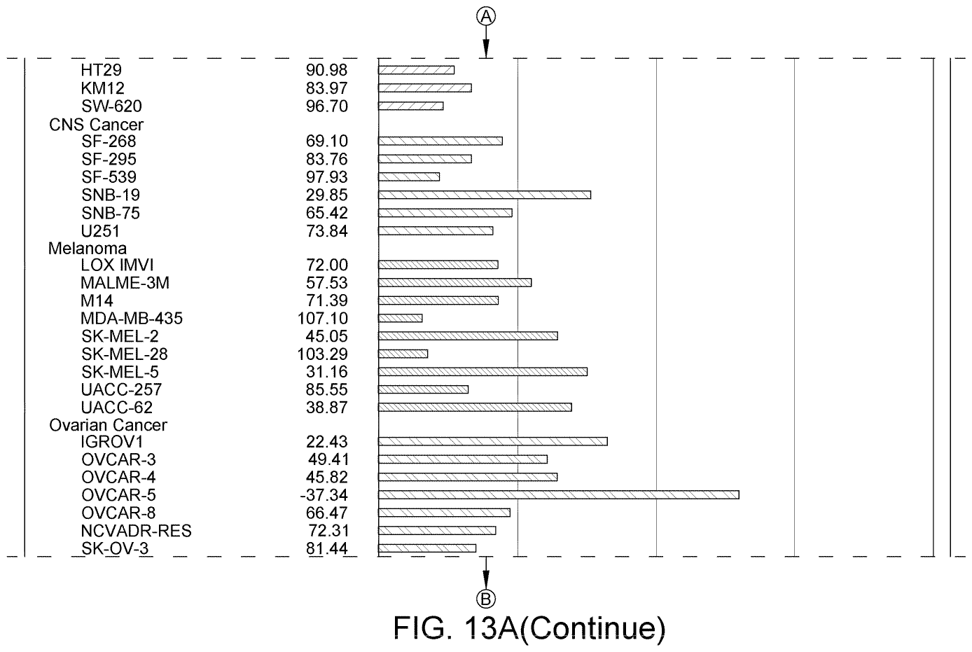

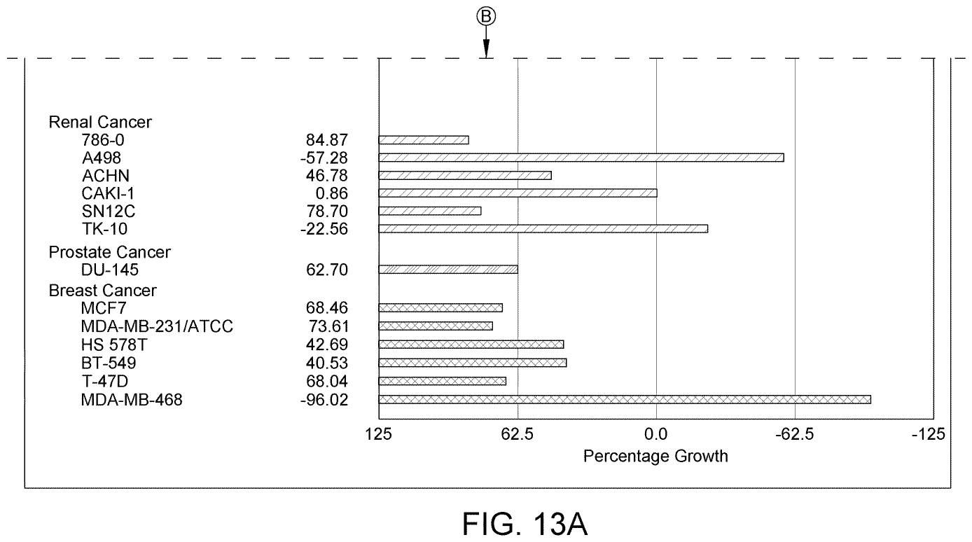

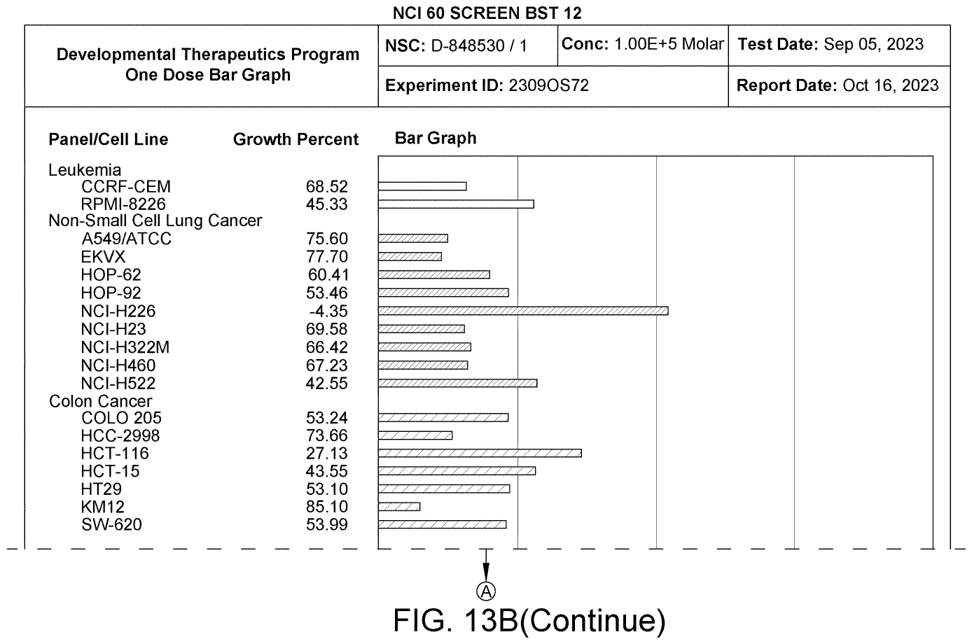

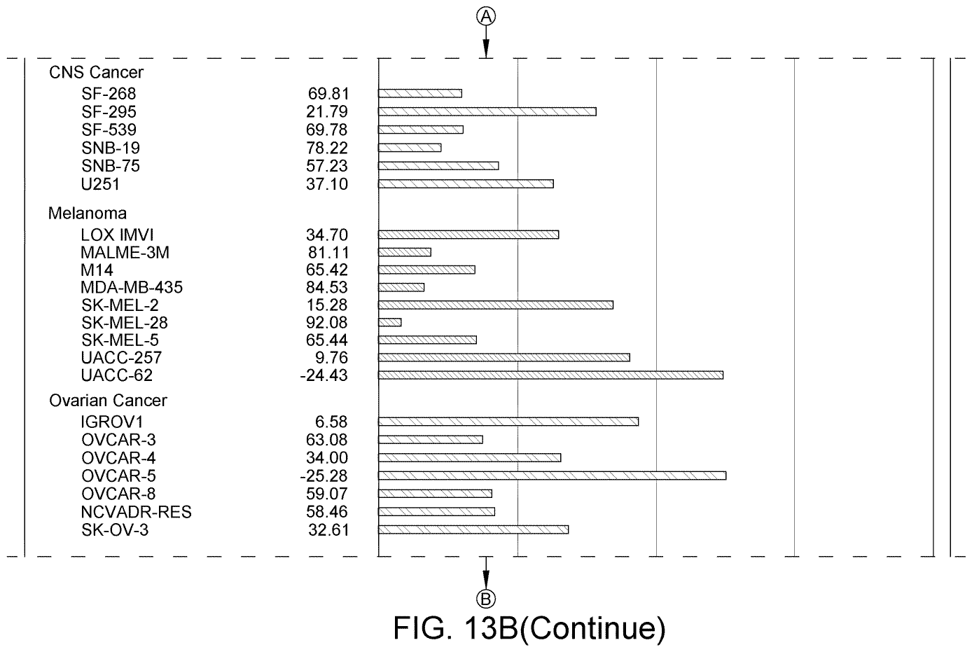

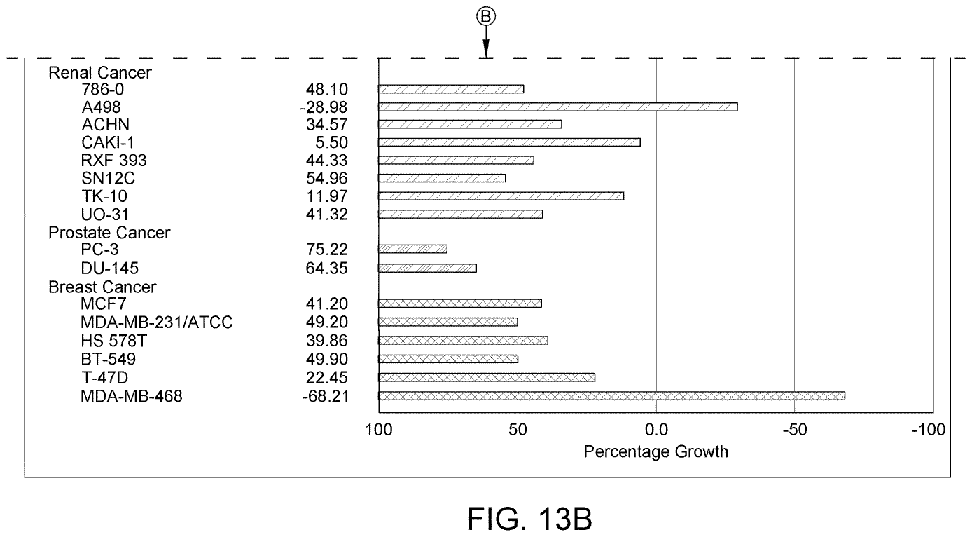

FIG. 13A shows using NCI-60 screening of 10 μM BST10 against 60 cell lines used to identify anti-cancer drugs. Seven cell lines sensitive to BST10 were identified: Leukemia Cell Lines MOLT-4 and SR; Non-Small Cell Lung Cancer Cell Line NCI-H226; Ovarian Cancer Cell Line OVCAR-8; and, Renal Cancer Cell Lines A498, CAKI-1 and TK-10.

FIG. 13B shows using NCI-60 screening of 10 μM BST12 against the same 60 cell lines used to identify anti-cancer drugs. Five cell lines sensitive to BST12 were identified: Non-Small Cell Lung Cancer Cell Line NCI-H226; Melanoma Cell Line UACC-62; Ovarian Cancer Cell Line OVCAR-5; Renal Cancer Cell Lines A498; and, Breast Cancer Cell Line MDA-MB-468.

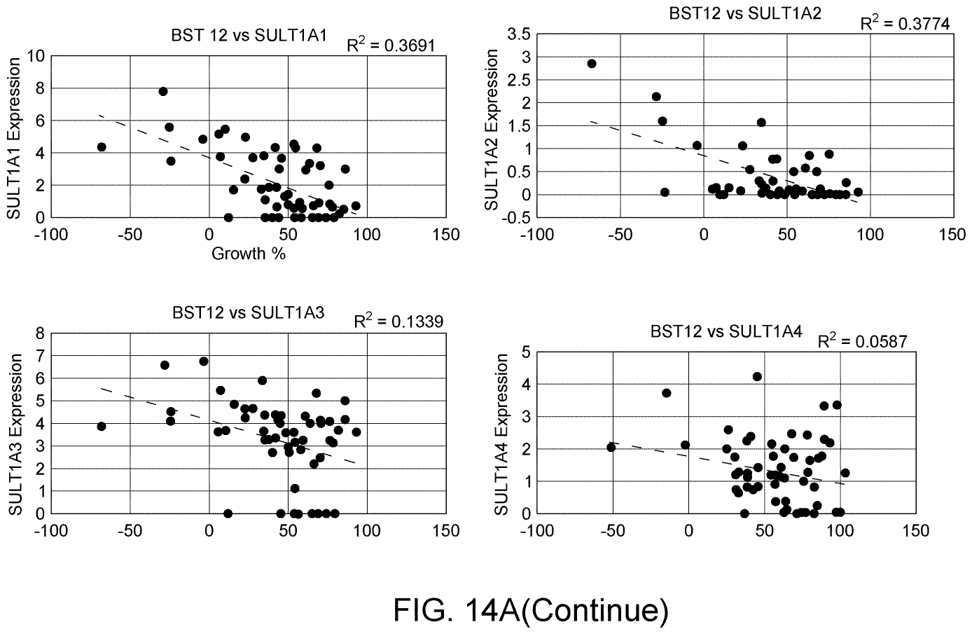

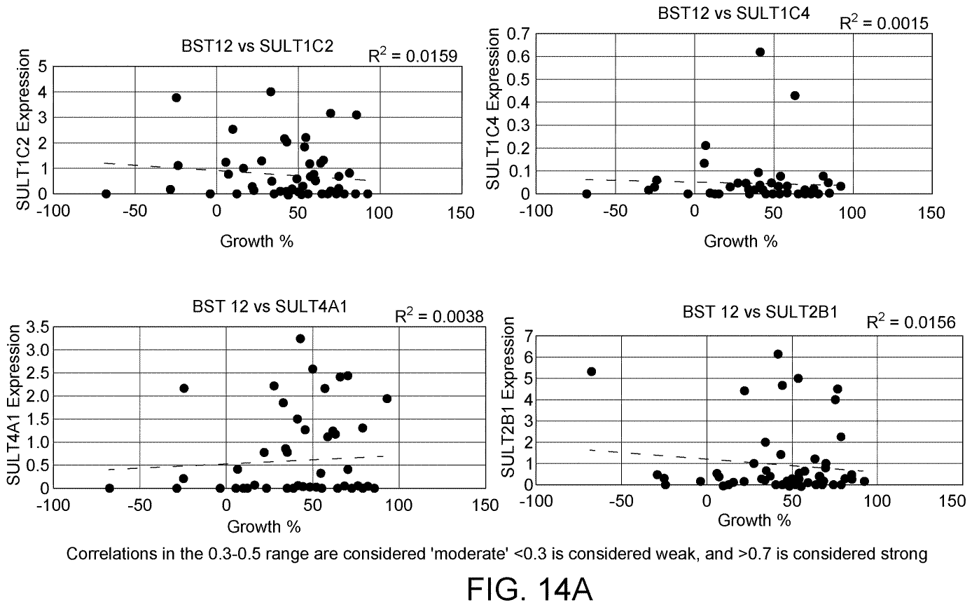

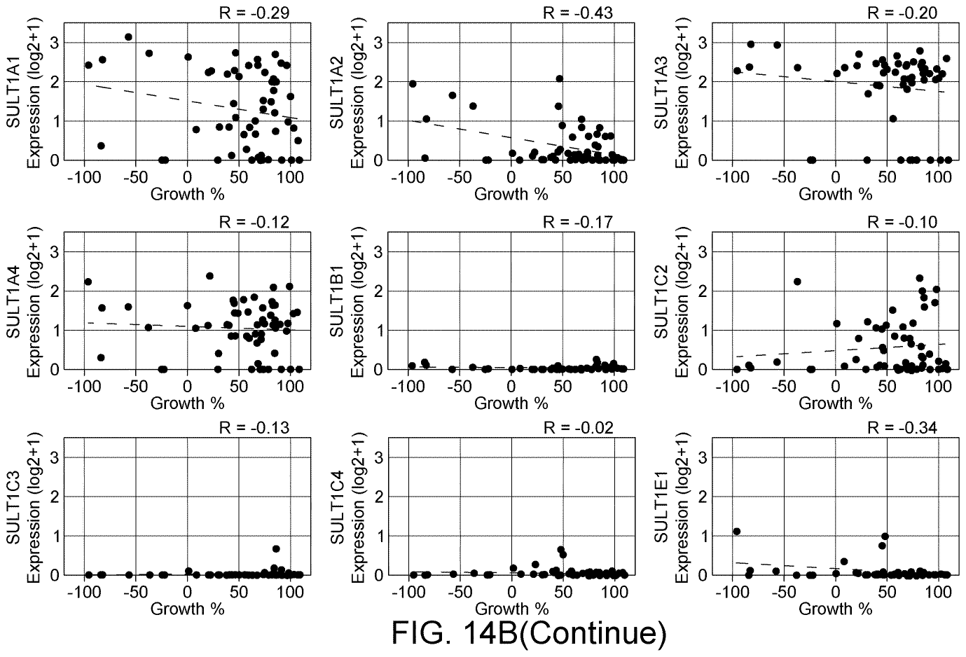

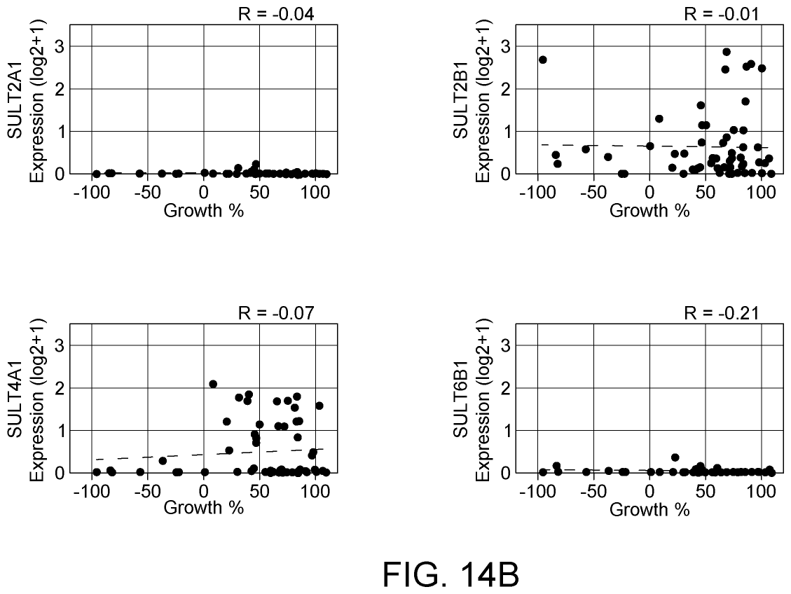

FIGS. 14A & 14B show SULT isoform expression data from DepMap analyzed in relation to the growth inhibition of NCI-60 cell lines treated with BST12 (FIG. 14A) and BST10 (FIG. 14B). The results show a moderate to strong negative correlation between growth and SULT1A1 expression, indicating that SULT1A1-positive cell line growth was inhibited by BST10 and BST12 treatment. Each data point represents the percentage of growth inhibition following a single 10 μM dose of BST10 or BST12 in a cancer cell line. mRNA expression data is sourced from the DepMap database. The Pearson correlation coefficient is represented as R and R². Correlation in the 0.3 to 0.5 range is considered moderate; less than 0.3 is considered weak, and more than 0.7 is considered strong.

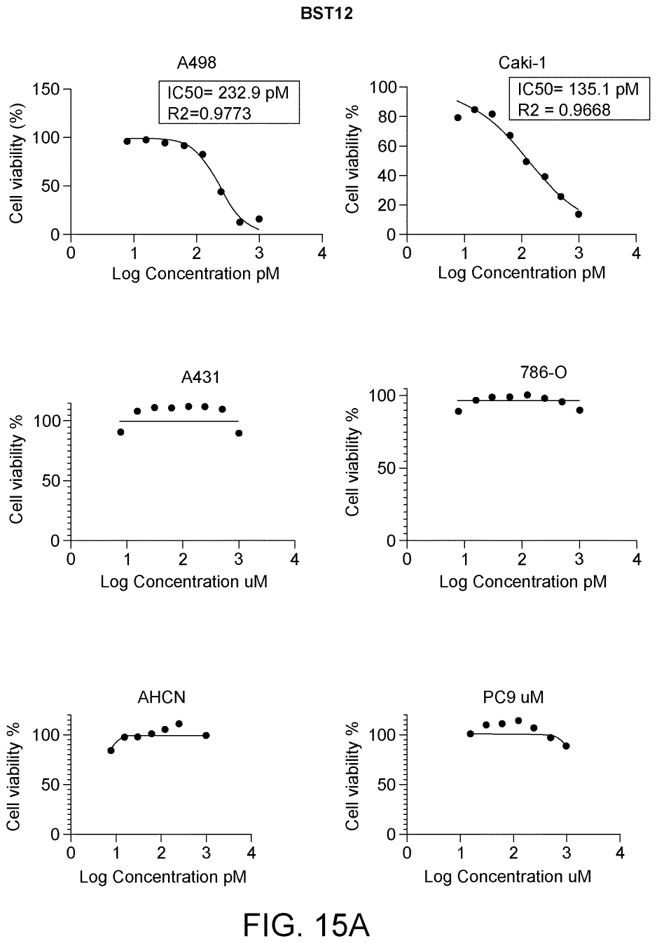

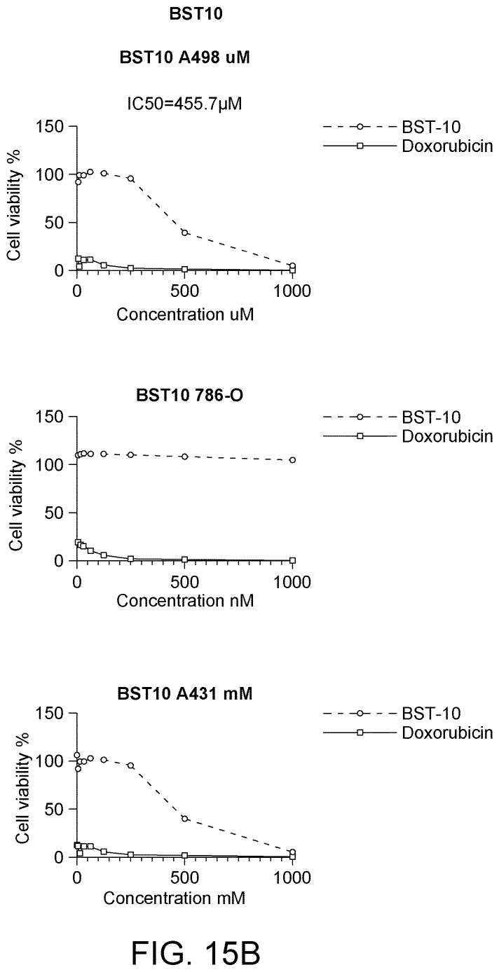

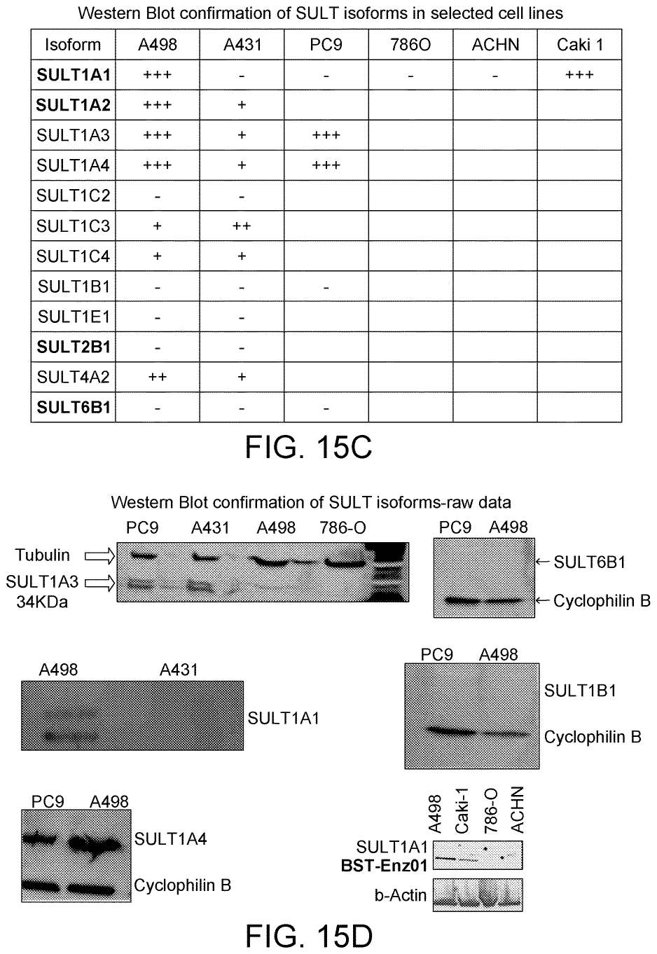

In vitro cytotoxicity of BST12 (FIG. 15A) and BST10 (FIG. 15B) was assessed using the Cell Titer-Glo assay in SULT1A1-positive and negative cell lines. The results confirm that only SULT1A1-positive cell line A498 (verified as SULT1A1-positive in FIG. 15C and FIG. 15D) is highly susceptible to both BST10 and BST12-induced cytotoxicity, with IC₅₀ values in the picomolar range for BST12 and in the micromolar range for BST10. The SULT1A1-positive cell lines A498 and Cakl-1 were sensitive to BST12 and similarly the cell line A498 was sensitive to BST10.

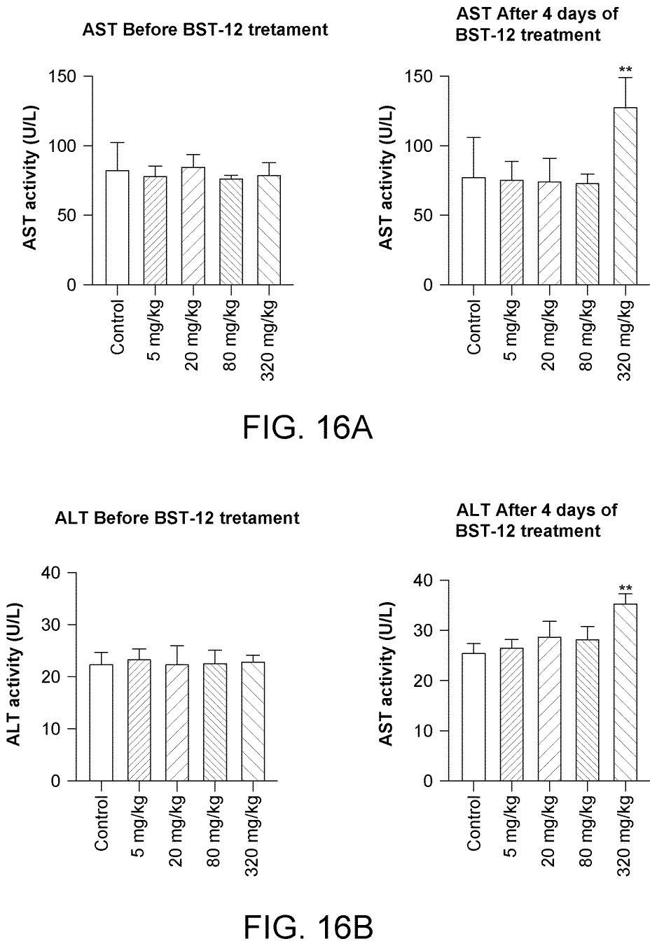

FIGS. 16A and 16B show the results for Sprague Dawley rats (200-250 g) administered BST12 daily via intraperitoneal injection for 5 days across 5 dosage groups (n=4 per group). Serum ALT and AST levels were measured on days 0, 2, and 6 to assess liver toxicity. FIG. 16A shows that Aspartate Aminotransferase (AST), a marker for both muscle and liver damage, remained within reference ranges (37-205 U/L) except at the highest dose (320 mg/kg), where a significant increase (p<0.05) was observed. FIG. 16B shows that Alanine Aminotransferase (ALT), a liver-specific marker (reference: 6-114 U/L), showed a significant increase (p<0.05) at 320 mg/kg after 4 days, with a non-significant upward trend at 20 and 80 mg/kg.

These results demonstrate that BST-10, BST-12 and Compound 103 can be effective cancer treatments for certain cancers, with minimal toxicity.

It is to be noted that various modifications are possible to provide additional functionalities to the embodiments of suture passing instrument described above. It is to be understood that the foregoing description and embodiments are intended to merely illustrate and not limit the scope of the invention. Other embodiments, modifications, variations and equivalents of the invention are apparent to those skilled in the art and are also within the scope of the invention, which is only described and limited in the claims which follow, and not elsewhere.

What is claimed is:

1. A therapeutic composition including one or more of the compounds below:

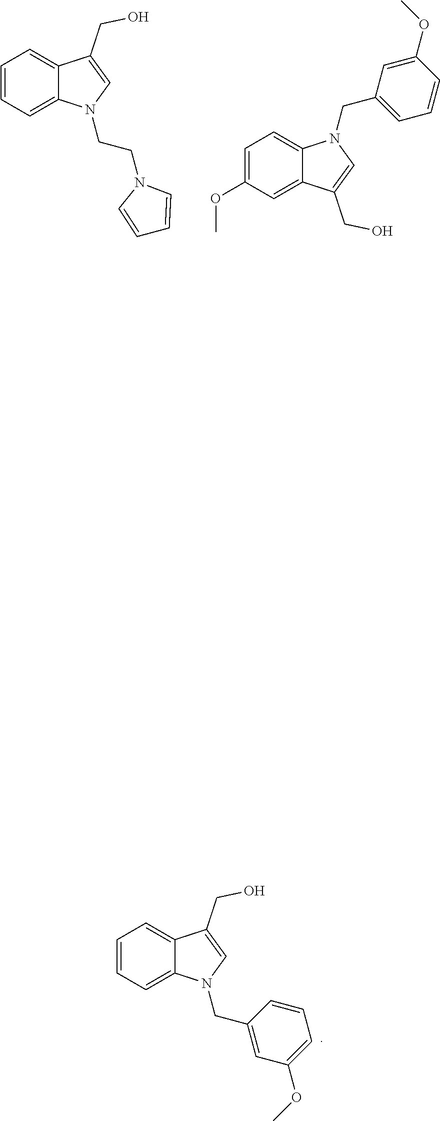

2. The composition of claim 1 further including a cyclodextrin.

3. The composition of claim 2 wherein the cyclodextrin is a β-cyclodextrin or a γ-cyclodextrin.

4. The composition of claim 2 wherein the cyclodextrin is methyl-β-cyclodextrin, hydroxypropyl-γ-cyclodextrin, hydroxypropyl-β-cyclodextrin or sulfobutylether-β-cyclodextrin.

5. The composition of claim 1 further including a pharmaceutically acceptable carrier.

6. The composition of claim 5 where the carrier includes polyethylene glycol (15)-hydroxystearate.

7. The composition of claim 5 wherein the carrier includes phosphate buffered saline.

8. The composition of claim 5 wherein the carrier includes 30% acetonitrile.

9. A method of treating a disease in a subject in need thereof, said method comprising of administering to said subject a pharmaceutical composition of claim 1.

10. The method of claim 8, where in the disease is a cancer.
